SEQUENCE LISTING (16 sequences)

SEQ ID NO: 1 - DNA, 10 bp, Artificial Sequence
  Description of Artificial Sequence XmnI restriction site
gaannnnttc

SEQ ID NO: 2 - DNA, 101 bp, Artificial Sequence
  Description of Artificial Sequence Synthetic DNA fragment
acgcgtacattgacatatataaacccgcctcctccttgtttagggtttctacgtgagagaagacgaaacacccaagacgcagatccccatcgaattcgatc

SEQ ID NO: 3 - DNA, 25 bp, Artificial Sequence
  Description of Artificial Sequence Primer
catgatgtggtggcactaattgtac

SEQ ID NO: 4 - DNA, 25 bp, Artificial Sequence
  Description of Artificial Sequence Primer
ctgtcaggcgtgtttttccagccac

SEQ ID NO: 5 - DNA, 25 bp, Artificial Sequence
  Description of Artificial Sequence Primer
tgattggcccggaaggcggtttatc

SEQ ID NO: 6 - DNA, 25 bp, Artificial Sequence
  Description of Artificial Sequence Primer
tcagagtctcaacgagatccttctc

SEQ ID NO: 7 - DNA, 29 bp, Artificial Sequence
  Description of Artificial Sequence Primer
cgggaggtcagcatgctcccggacgtatc

SEQ ID NO: 8 - DNA, 29 bp, Artificial Sequence
  Description of Artificial Sequence Primer
cgggaggtcagcatgctcccggacgtatc

SEQ ID NO: 9 - DNA, 24 bp, Artificial Sequence
  Description of Artificial Sequence Oligo
gaagtgagaaccatggcttctatg

SEQ ID NO: 10 - DNA, 22 bp, Artificial Sequence
  Description of Artificial Sequence Oligo
cgcgcataaaggtcagcatatg

SEQ ID NO: 11 - DNA, 27 bp, Artificial Sequence
  Description of Artificial Sequence Oligonucleotide
accgtcgacgagctcgtacggtatcga

SEQ ID NO: 12 - DNA, 29 bp, Artificial Sequence
  Description of Artificial Sequence Oligonucleotide
tcgatcgataccgtacgagctcgtcgacg

SEQ ID NO: 13 - DNA, 11 bp, Artificial Sequence
  Description of Artificial Sequence Oligonucleotide
tcgacgagctc

SEQ ID NO: 14 - DNA, 29 bp, Artificial Sequence
  Description of Artificial Sequence Primer
cgggaggtcaccatggtcccggacgtatc

SEQ ID NO: 15 - DNA, 27 bp, Artificial Sequence
  Description of Artificial Sequence Primer
cggagaagaaccatggtcaagctcggc

SEQ ID NO: 16 - DNA, 10 bp, Artificial Sequence
  Description of Artificial Sequence ClaI linker
gcatcgatgc

(19) United States
(12) Patent Application Publication
(10) Pub. No.: US 2004/0052774 A1
(43) Pub. Date: Mar. 18, 2004

(54) Reducing oxidative stress of plants by increasing glutathione content

(76) Inventors: Gary Patrick Creissen, Norwich (GB); Philip Mark Mullineaux, Norwich (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/399,313

(22) PCT Filed: Oct. 12, 2001
(86) PCT No.: PCT/GB01/04559

(30) Foreign Application Priority Data
Oct. 16, 2000 (GB) 0025312.0

(51) Int. Cl. A61K 48/00; C12N 15/85

(52) U.S. Cl. 424/93.21; 435/455; 435/320.1

(57) ABSTRACT

Disclosed are stable recombinant multi-gene nucleic acid constructs, such as plant binary vectors, comprising (i) a gene encoding γ-glutamylcysteine synthetase and (ii) a gene encoding glutathione synthetase, plus preferably at least one, preferably two, genes which encode enzymes involved in the redox cycling of glutathione between its reduced and its oxidised forms e.g. glutathione reductase and/or glutathione peroxidase. Preferably the promoters linked to the genes are different and of different strengths, and may optionally be inducible. Also provided are related materials and corresponding methods and uses e.g. in plants to improve oxidative stress tolerance enhance root development, or to increase the post-harvest shelf life of the plant or part thereof.

### TECHNICAL FIELD

The present invention relates generally to methods and materials for use in modifying plant characteristics. In particular, the present invention relates to novel methods and materials for reducing oxidative stress in plants.

### BACKGROUND TO THE INVENTION

Plants possess an array of compounds which have antioxidant properties and which are believed to be important in the protection against a variety of abiotic and biotic stresses. These include glutathione (γ-L-glutamyl-L-cysteinyl-L-glycine [GSH]), ascorbic acid (vitamin C), phenolic isoflavanoid compounds, α-tocopherol (vitamin E), and the carotenoids, including the xanthophylls (Fryer (1993) Plant Cell Environ 15 381-392; Mullineaux and Creissen (1996) Biochem Soc Trans 24, 829-835). The reduced forms of these compounds together with antioxidant enzymes are believed to scavenge reactive oxygen species (ROS) and other products of oxidative reactions. Such antioxidant enzymes include superoxide dismutase, catalase, ascorbate peroxidase (APX), glutathione peroxidase (GPX), glutathione S-transferase/glutathione peroxidase (GST/GPX), dehydroascorbate reductase, monodehydroascorbate free radical reductase, and glutathione reductase (GOR).

Several reduction-oxidation (redox) cycles that scavenge ROS in different subcellular compartments and that involve these enzymes and antioxidants have been proposed (e.g. the ascorbate-GSH cycle). The reducing equivalents for these reactions are derived ultimately from photosynthetic electron transport (Foyer and Halliwell (1976) Planta 133, 21-25; Mullineaux and Creissen (1997) Oxidative Stress and the Molecular Biology of Antioxidant Defences (J. Scandalios, ed. Cold Spring Harbor Laboratory Press) pp 667-713). Therefore the degree of reduction of major antioxidant pools is generally considered to reflect the redox status of the tissue in question and is consequently an indicator of oxidative stress.

Glutathione, either as GSH or as GSSG (glutathione disulfide; oxidised glutathione) is regarded as a key component of antioxidant defences in most aerobic organisms, including plants (Foyer et al., 1997). GSH is synthesized from its constituent amino acids in an ATP-dependent two step reaction catalyzed by the enzymes γ-glutamylcysteine synthetase (γ-ECS; EC 6.3.2.2) and glutathione synthetase (GS; EC 6.3.2.3). In plants, GSH biosynthesis occurs in the cytosol and the chloroplast, with at least one control point being the regulation of activity of γ-ECS (Hell and Bergmann (1990) Planta 180 603-612; Ruegsegger and Brunold (1993) Plant Physiol. 101 561-566; (Noctor et al.(1996) Plant physiol. 112 1071-1078), Noctor et al.(1997) Physiol Plant. 100, 255-263)). Additional regulation of GSH biosynthesis may be achieved by the supply of its constituent amino acids (Strohm et al., (1995) Plant J 7, 141-145; Noctor et al., 1997, supra).

Foliar GSH levels have been successfully raised by three- to fourfold in poplar transformed with the coding sequence of the γ-ECS gene (gshI) from Escherichia coli under the control of the cauliflower mosaic virus (CaMV) 35S promoter (Noctor et al., 1996 Plant physiol. 112 1071-1078). Conversely, poplar transformants overexpressing a transgene encoding E. coli GS (gsh2) did not show any increase in foliar GSH content (Strohm et al., 1995 Plant J 7, 141-145). Overexpression of the first committed step of glutathione biosynthesis in chloroplasts of transgenic tobacco (cpGSHI plants) paradoxically resulted in increased oxidative stress (Creissen et al (1999) Plant Cell 11, 1277-1291). This was related to the accumulation of oxidised γ-glutamylcysteine (bis-γ-glutamylcystine) and it was proposed that plants accumulating this compound suffered a failure in redox sensing. Crossing with plants which expressed GS in the chloroplast (cpGSHII plants) resulted in an increase in the redox status of the bis-γ-glutamylcystine pool and an anmelioration of the oxidative stress symptoms (Creissen et al 1999, supra).

Glutathione has a specific role in the reduction of hydrogen peroxide and lipid peroxides via the reactions

2GSH+H₂O₂→GSSG+2H₂O

2GSH+2ROOH→GSSG+ROH+H₂O,

GSSG is recycled back to the reduced form GSH by the action of glutathione reductase (GOR). Manipulation of the glutathione reductase levels in transgenic tobacco (Broadbent et al 1995) was shown to provide increased tolerance to certain oxidative stresses; however the results were not found to be fully reproducible between lines. There are at least two forms of the GOR gene—GOR1 (encoding plastidial glutathione reductase) and GOR2 (encoding cytosolic glutathione reductase).

There are two forms of glutathione peroxidase enzyme in plants—the chloroplastic glutathione peroxidase and the cytosolic glutathione peroxidase. The cytosolic glutathione peroxidase is believed to have two functions—as a glutathione peroxidase and as a glutathione —S-transferase and so is known as GPX/GST. In contrast, the chloroplast glutathione peroxidase does not have S-transferase activity and so is known simply as GPX.

### DISCLOSURE OF THE INVENTION

The present inventors have discovered that a significant improvement in oxidative stress tolerance may be achieved by manipulating at least two of the enzymes involved in the synthesis of glutathione such that the two enzymes are differentially expressed, and, optionally, at least one enzyme involved in the glutathione redox cycle. Moreover, the inventors have overcome considerable technical difficulties in providing stable multi-gene DNA constructs to provide a multi-gene DNA construct which enables the stable transformation of a plant cell. In preferred examples, three or four separate genes which are together involved in the glutathione synthesis and turnover pathways are present on a single construct.

Multi-gene DNA constructs of the invention which include the firefly luciferase (luc) reporter gene at the right T-DNA border and kanamycin resistance (kan; NPTII) selectable marker at the left border have been used to identify transgenic plants of two crop species, namely tomato and lettuce, which express several genes associated with glutathione metabolism either in the chloroplast or in the cytosol.

As described below, plants which express these genes in either or both subcellular compartments have increased glutathione content in the leaves and/or in developing fruit. Furthermore this capacity is maintained in progeny derived from self-pollination of the primary transgenics and shows clear segregation from azygous progeny (which have not inherited the transgene). Therefore this increased capacity for glutathione biosynthesis is a direct consequence of transgene expression.

The increased antioxidant capacity resulting from sustained elevated glutathione content is expected to have a number of benefits for the plant and the post-harvest product (leaf, fruit, seed etc). Such benefits include resistance to a number of potentially damaging oxidative events arising from both biotic and abiotic stresses. Furthermore it is now well established that GSH plays an important role in development. Plants with a mutation in one of the enzymes involved in glutathione synthesis (-glutamylcysteine synthetase) are almost devoid of glutathione (less than 3% of wild-type levels) and show inhibition of root cell division (Vernoux et al 200; Plant Cell 12, 97-109). In addition, progression through the cell cycle in tobacco cell suspension culture is dependent on an adequate GSH concentration (Vernoux et al; ibid).

Therefore the methods and vectors of the present invention may be used to increase tolerance to biotic and abiotic stresses in plants and enhance capacity for antioxidant regeneration; alter root development leading to increased root mass and consequent improvements in water use and nutrient uptake; and improve shelf life of post-harvest products and therefore find use in a number of important crop species, for instance during growth of a plant and/or during post-harvest storage product of the plant (e.g tomato, pepper, aubergine, courgette), leaves (e.g. lettuce, cabbage) flowers (e.g. broccoli, ornamentals), storage organs (potato, yam) and seeds (e.g grains, pulses).

Thus according to one aspect of the present invention there is provided a method of manipulating the oxidative status of a plant comprising introducing into the plant a nucleic acid construct which encodes γ-ECS (γ-glutamylcysteine synthetase) and GS (glutathione synthetase), wherein the genes encoding γ-ECS and GS are differentially expressed under the control of different promoters. Optionally, the construct also includes at least one gene encoding an enzyme involved in the redox cycling of glutathione between its reduced and its oxidised forms. Preferably, the gene encoding an enzyme involved in the redox cycling of glutathione will be GOR (encoding glutathione reductase) or a gene encoding a glutathione peroxidase (GPX or GPX/GST). Most preferably, the nucleic acid construct will comprise GOR and a glutathione peroxidase gene. The alteration in the oxidative status may be assessed by comparison with a plant in which the nucleic acid has not been so introduced. Preferably the construct is comprised within a vector.

Where reference is made to gsh1, gsh2 or genes encoding an antioxidant enzyme capable of reducing oxidised glutathione, such as GOR or other genes involved in the glutathione redox cycle, it should be understood that, except where the context demands otherwise, variants, both natural and artificial, may be used as long as the variant forms retain the ability to encode a polypeptide with an appropriate corresponding enzymatic capability. For example, gsh1 may be substituted by any nucleic acid which encodes an enzyme retaining a γ-glutamylcysteine synthetase activity. Further, where reference is made to GOR, it should be understood that, except where the context dictates otherwise, GOR1 or GOR2 may be used.

A variant nucleic acid molecule shares homology with, or is identical to, all or part of at least one of the nucleotide sequences of the genes discussed above. Variant nucleic acids may include a sequence encoding a functional polypeptide (e.g. which is a variant of gsh1, gsh2 or GOR and which may cross-react with an antibody raised to said polypeptide). Generally variants may be used to alter the oxidative stress resistance characteristics of plants as described above. Alternatively they may include a sequence which interferes with the expression or activity of such a polypeptide (e.g. sense or anti-sense suppression).

Generally speaking variants may be naturally occurring nucleic acids, or they may be artificial nucleic acids. Variants may include orthologues, alleles, isoalleles or homologues of any one of the gsh1, gsh2, gor, gpx or gpx/gst genes. Particularly included are variants which include only a distinctive part or fragment (however produced) corresponding to a portion of the relevant gene, encoding at least functional parts of the polypeptide. Suitable lengths of fragment, and conditions, for such processes are discussed in more detail below. Also included are nucleic acids corresponding to those above, but which have been extended at the 3′ or 5′ terminus. The term ‘variant’ nucleic acid as used herein encompasses all of these possibilities. Except where the context demands otherwise, where reference is made herein to any one of gsh1, gsh2, gor, gpx or gpx/gst, such a reference should be understood to include reference to variants of the appropriate gene. Vectors which are variants of those disclosed herein will have the essential properties of them as described herein, for example be stable and capable of modifying the production and\or redox cycling of glutathione in organisms in which they are expressed. Where vectors or constructs are said to comprise one or more of these genes they may in preferred forms consist essentially of them i.e. not include other genes unrelated to either the glutathione function or the function and\or selectable properties of the vector itself.

Similarity or homology may be as defined and determined by the TBLASTN program, of Altschul et al. (1990) J. Mol. Biol. 215: 403-10, or BestFit, which is part of the Wisconsin Package, Version 8, September 1994, (Genetics Computer Group, 575 Science Drive, Madison, Wis., USA, Wisconsin 53711). Preferably sequence comparisons are made using FASTA and FASTP (see Pearson & Lipman, 1988. Methods in Enzymology 183: 63-98). Parameters are preferably set, using the default matrix, as follows:

Gapopen (penalty for the first residue in a gap): −12 for proteins/−16 for DNA

Gapext (penalty for additional residues in a gap): −2 for proteins/−4 for DNA

KTUP word length: 2 for proteins/6 for DNA.

Homology may be at the nucleotide sequence and/or encoded amino acid sequence level. Preferably, the nucleic acid and/or amino acid sequence shares at least about 60%, or 70%, or 80% homology, most preferably at least about 90%, 95%, 96%, 97%, 98% or 99% homology. Homology may be over the full-length of the relevant sequence, or may be over a part of it, preferably over a contiguous sequence of about or greater than about 20, 25, 30, 33, 40, 50, 67, 133, 167, 200, 233, 267, 300, 333, 400 or more amino acids or codons, compared with the appropriate known sequence.

Thus a variant polypeptide may include a single amino acid change, or 2, 3, 4, 5, 6, 7, 8, or 9 changes, about 10, 15, 20, 30, 40 or 50 changes, or greater than about 50, 60, 70, 80 or 90 changes. In addition to one or more changes within the amino acid sequence shown, a variant polypeptide may include additional amino acids at the C-terminus and/or N-terminus. Naturally, changes to the nucleic acid which make no difference to the encoded polypeptide (i.e. ‘degeneratively equivalent’) are included.

Alternatively changes to a sequence may produce a derivative by way of one or more of addition, insertion, deletion or substitution of one or more nucleotides in the nucleic acid, leading to the addition, insertion, deletion or substitution of one or more amino acids in the encoded polypeptide.

Such changes may modify sites which are required for post translation modification such as cleavage sites in the encoded polypeptide; motifs in the encoded polypeptide for glycosylation, lipoylation etc. Leader or other targeting sequences (e.g. membrane or golgi locating sequences) may be added to the expressed protein to determine its location following expression.

Other desirable mutations may be made at random or via site directed mutagenesis in order to alter the activity (e.g. specificity) or stability of the encoded polypeptide. Changes may be by way of conservative variation, i.e. substitution of one hydrophobic residue such as isoleucine, valine, leucine or methionine for another, or the substitution of one polar residue for another, such as arginine for lysine, glutamic for aspartic acid, or glutamine for asparagine. As is well known to those skilled in the art, altering the primary structure of a polypeptide by a conservative substitution may not significantly alter the activity of that peptide because the side-chain of the amino acid which is inserted into the sequence may be able to form similar bonds and contacts as the side chain of the amino acid which has been substituted out. This may be so even when the substitution is in a region which is critical in determining the conformation of a peptide. Also included are variants having non-conservative substitutions. As is well known to those skilled in the art, substitutions to regions of a peptide which are not critical in determining its conformation may not greatly affect its activity because they may not significantly alter the three dimensional structure of the peptide. In regions that are critical in determining the conformation or activity of the peptide such changes may confer advantageous properties on the polypeptide. Indeed, changes such as those described above may confer slightly advantageous properties on the peptide e.g. altered stability or specificity.

The homology between nucleic acid sequences may be determined with reference to the ability of the nucleic acid sequences to hybridise to each other. Preliminary experiments may be performed by hybridising under low stringency conditions. For probing, preferred conditions are those which are stringent enough for there to be a simple pattern with a small number of hybridisations identified as positive which can be investigated further.

One common formula for calculating the stringency conditions required to achieve hybridization between nucleic acid molecules of a specified sequence homology is (Sambrook et al., 1989):

Tₘ=81.5° C.+16.6 Log [Na+]+0.41 (% G+C)−0.63 (% formamide)−600/#bp in duplex

As an illustration of the above formula, using [Na+]=[0.368] and 50-% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. Such a sequence would be considered substantially homologous to nucleic acid sequences used in the present invention.

It is well known in the art to increase stringency of hybridisation gradually until only a few positive clones remain. Other suitable conditions include, e.g. for detection of sequences that are about 80-90% identical, hybridization overnight at 42° C. in 0.25M Na₂HPO₄, pH 7.2, 6.5% SDS, 10% dextran sulfate and a final wash at 55° C. in 0.1×SSC, 0.1% SDS. For detection of sequences that are greater than about 90% identical, suitable conditions include hybridization overnight at 65° C. in 0.25M Na₂HPO₄, pH 7.2, 6.5% SDS, 10% dextran sulfate and a final wash at 60° C. in 0.1×SSC, 0.1% SDS.

In a further aspect of the invention there is provided a recombinant multi-gene nucleic acid construct, comprising the gsh1 gene (encoding γ-glutamylcysteine synthetase) and the gsh2 gene (encoding glutathione synthetase) wherein the gsh1 gene and the gsh2 gene are under the control of different promoters to enable differential expression of γ-glutamylcysteine synthetase and glutathione synthetase. Optionally, the construct also comprises at least one gene encoding an enzyme involved in the redox cycling of glutathione between its reduced and its oxidised forms. The recombinant multi-gene construct of the invention is preferably provided as a recombinant vector. In a preferred embodiment, the construct will comprise gsh1 and gsh2 with gsh1 under the control of a weaker promoter, that is to say a weaker promoter compared with the promoter controlling gsh2 to drive expression of glutathione synthetase. Preferably the construct will comprise gsh1, gsh2 and GOR genes. In a more preferred embodiment the construct will comprise a gsh1 gene, a gsh2 gene, a GOR gene and a glutathione peroxidase encoding gene. In one preferred vector of the invention the construct comprises gsh1 (encoding γ-glutamylcysteine synthetase), gsh2 (encoding glutathione synthetase), GOR1 (encoding plastidial glutathione reductase) and phGPX (encoding phospolipid hydroperoxide glutathione peroxidase) genes. In another preferred vector of the invention the construct comprises gsh1, gsh2, GOR2 (encoding cytosolic glutathione reductase) and GST/GPX (encoding cytosolic glutathione peroxidase/glutathione-S-transferase) genes.

“Vector” is defined to include, inter alia, any plasmid, cosmid, phage or Agrobacterium binary vector in double or single stranded linear or circular form which may or may not be self transmissible or mobilizable, and which can transform a prokaryotic or eukaryotic host either by integration into the cellular genome or exist extrachromosomally (e.g. autonomous replicating plasmid with an origin of replication). Preferably the vector is a plasmid.

Generally speaking, those skilled in the art are well able to construct vectors and design protocols for recombinant gene expression. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, 1992.

Specifically included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells).

As stated above, the nucleic acid in a vector is under the control of, and operably linked to, at least two appropriate promoters or other regulatory elements for transcription in a host cell such as a plant cell, with each of gsh1 and gsh2 operably linked to a different promoter. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell. By “promoter” is meant a sequence of nucleotides from which transcription may be initiated of DNA operably linked downstream (i.e. in the 3′ direction on the sense strand of double-stranded DNA). “Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment, at least one of the promoters is an inducible promoter. The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus.

Thus this aspect of the invention provides a replicable vector according to the invention, wherein at least one of the promoters is inducible, and operably linked to one of gsh1, gsh2, and/or a gene encoding an enzyme involved in the redox cycling of glutathione between its reduced and its oxidised forms, for example a GOR, GPX or GPX/GST gene. Preferably the coding sequence with which a promoter is operably linked is not the same coding sequence with which it is operably linked in nature.

The present invention also provides methods comprising introduction of such a replicable vector, wherein at least one promoter is inducible, into a plant cell, and/or induction of expression of a construct within the plant cell, by application of a suitable stimulus e.g. an effective exogenous inducer.

Specific procedures and vectors previously used with wide success upon plants are described by Guerineau and Mullineaux (1993) (Plant transformation and expression vectors. In: Plant Molecular Biology Labfax (Croy RRD ed) Oxford, BIOS Scientific Publishers, pp 121-148), the teaching of which is herein incorporated by reference. Suitable vectors may include plant viral-derived vectors (see e.g. EP-A-194809).

Suitable promoters which operate in plants include the Cauliflower Mosaic Virus 35S (CaMV 35S). Other examples are disclosed at pg 120 of Lindsey & Jones (1989) “Plant Biotechnology in Agriculture” Pub. OU Press, Milton Keynes, UK, the teaching of which is herein incorporated by reference. The promoter may be selected to include one or more sequence motifs or elements conferring developmental and/or tissue-specific regulatory control of expression. Inducible plant promoters include the ethanol induced promoter of Caddick et al (1998) Nature Biotechnology 16: 177-180.

However, although the construction of vectors for expression of single recombinant genes have been known for many years, it is technically problematic to produce stable vectors comprising multi-gene constructs for use in plants. The present inventors have overcome this difficulty and have found that the stability of the multi-gene DNA constructs of the invention may be considerably improved by including in the recombinant vector a minimum number of repeated sequences, for example a minimum number of the same promoter sequences. In particular, it has been found that deleterious effects may be avoided by using different promoters operably linked to each of gsh1 and gsh2 to enable different levels of expression of γ-ECS and GS respectively. In a preferred embodiment, the level of expression of γ-ECS is not as high as the level of expression of GS under the control of a stronger promoter.

Therefore at least two different promoters, each of which is operably linked to a different gene involved in glutathione synthesis is used. Optionally at least on further promoter operably linked to a gene involved in glutathione cycling, where such a gene is present, may also be included. Preferably three different promoters and more preferably four different promoters are used, each of which is operably linked to a different gene of the construct.

Therefore, in a further aspect of the invention, there is provided a recombinant vector of the invention further comprising at least three different promoters each of which is operably linked to a different gene of the construct. Each of said gsh1 gene and gsh2 gene is operably linked to a different promoter and, preferably, when present, each gene encoding an enzyme involved in the redox cycling, e.g. GOR. GPX or GPX/GST is operably linked to a different promoter.

In a preferred embodiment, the gsh1 gene is operably linked to a Ef1a promoter (Liboz et al. Plant Mol. Biol. 14:107-110(1989)); the gsh2 gene is operably linked to a cauliflower mosaic virus (CaMV) 35S promoter (Noctor et al., 1996); the GOR gene, where used, is operably linked to a AtrpL1 promoter (Santos MCG. PhD thesis, UEA (1995) and the GPX gene, where used, is operably linked to a UBQ1 promoter (Collin et al, 1990, J. Biol. Chem 265 12486-12493). The UBQ1 promoter is identical or virtually identical to the promoter represented in Arabidopsis BAC clone F2206 (accession number ATF2206).

If desired, selectable genetic markers may be included in the construct, that is to say those that may be used to confer selectable phenotypes such as resistance to antibiotics or herbicides (e.g. kanamycin, hygromycin, phosphinotricin, chlorsulfuron, methotrexate, gentamycin, spectinomycin, imidazolinones and glyphosate).

In the most preferred embodiment of the vector of the invention, the vector is the pAFQ70-1 plasmid as illustrated in FIG. 13 or the pAFQ70-2 plasmid as illustrated in FIG. 20, or a plasmid substantially homologous with the pAFQ70-1 plasmid or pAFQ70-2 plasmid. Preferably, the nucleic acid sequence of the plasmid shares at least about 60%, or 70%, or 80% homology, most preferably at least about 90 k, 95%, 96%, 97%, 98% or 99% homology with the pAFQ70-1 plasmid or pAFQ70-2 plasmid.

In a further aspect of the invention, there is disclosed a host cell containing a heterologous construct according to the present invention, especially a plant cell.

The term “heterologous” is used broadly in this aspect to indicate that the gene/sequence of nucleotides in question (e.g. encoding (γ-ECS), GS, GOR, GPX and/or GPX/GST) have been introduced into said cells of the plant or an ancestor thereof, using genetic engineering, i.e. by human intervention. A heterologous gene may replace an endogenous equivalent gene, i.e. one which normally performs the same or a similar function, or the inserted sequence may be additional to the endogenous gene or other sequence. Nucleic acid heterologous to a plant cell may be non-naturally occurring in cells of that type, variety or species. Thus the heterologous nucleic acid may comprise a coding sequence of or derived from a particular type of plant cell or species or variety of plant, placed within the context of a plant cell of a different type or species or variety of plant. A further possibility is for a nucleic acid sequence to be placed within a cell in which it or a homologue is found naturally, but wherein the nucleic acid sequence is linked and/or adjacent to nucleic acid which does not occur naturally within the cell, or cells of that type or species or variety of plant, such as operably linked to one or more regulatory sequences, such as a promoter sequence, for control of expression.

The host cell (e.g. plant cell) is preferably transformed by the construct, that is to say that the construct becomes established within the cell, altering one or more of the cell's characteristics and hence phenotype e.g. with respect to antioxidant capacity due to, for example, elevated glutathione content or enhanced glutathione cycling.

Nucleic acid can be introduced into plant cells using any suitable technology, such as a disarmed Ti-plasmid vector carried by Agrobacterium exploiting its natural gene transfer ability (EP-A-270355, EP-A-0116718, NAR 12(22) 8711-87215 1984), particle or microprojectile bombardment (U.S. Pat. No. 5,100,792, EP-A-444882, EP-A-434616) microinjection (WO 92/09696, WO 94/00583, EP 331083, EP 175966, Green et al. (1987) Plant Tissue and Cell Culture, Academic Press), electroporation (EP 290395, WO 8706614 Gelvin Debeyser) other forms of direct DNA uptake (DE 4005152, WO 9012096, U.S. Pat. No. 4,684,611), liposome mediated DNA uptake (e.g. Freeman et al. Plant Cell Physiol. 29: 1353 (1984)), or the vortexing method (e.g. Kindle, PNAS U.S.A. 87: 1228 (1990d) Physical methods for the transformation of plant cells are reviewed in Oard, 1991, Biotech. Adv. 9: 1-11.

Agrobacterium transformation is widely used by those skilled in the art to transform dicotyledonous species.

There has also been substantial progress towards the routine production of stable, fertile transgenic plants in almost all economically relevant monocot plants (see e.g. Hiei et al. (1994) The Plant Journal 6, 271-282)). Microprojectile bombardment, electroporation and direct DNA uptake are preferred where Agrobacterium alone is inefficient or ineffective. Alternatively, a combination of different techniques may be employed to enhance the efficiency of the transformation process, eg bombardment with Agrobacterium coated microparticles (EP-A-486234) or microprojectile bombardment to induce wounding followed by co-cultivation with Agrobacterium (EP-A-486233). The skilled person will appreciate that the particular choice of a transformation technology may be determined by its efficiency to transform certain plant species depending on the ease of use as well as the experience, preference and skill of the person practising the invention.

Thus a further aspect of the present invention provides a method of transforming a plant cell involving introduction of a vector as described above into a plant cell and causing or allowing recombination between the vector and the plant cell genome to introduce at least gsh1 and gsh2 into the genome, with gsh1 and gsh2 under the control of different promoters. Preferably the vector contains at least one further gene encoding an enzyme involved in the redox cycling of glutathione between its reduced and its oxidised forms will also be introduced into the genome.

The invention further encompasses a host cell, especially a plant cell, transformed with a vector according to the present invention (e.g. comprising the gsh1 gene (encoding -glutamylcysteine synthetase) and the gsh2 gene (encoding glutathione synthetase) under the control of different promoters to enable differential expression of γ-ECS and GS, and optionally comprising at least one gene encoding an enzyme involved in the redox cycling of glutathione between its reduced and its oxidised forms). In the transgenic plant cell (i.e. transgenic for the nucleic acid in question) the transgene may be on an extra-genomic vector or incorporated, preferably stably, into the genome. There may be more than one heterologous nucleotide sequence per haploid genome.

Generally speaking, following transformation, a plant may be regenerated, e.g. from single cells, callus tissue or leaf discs, as is standard in the art. Almost any plant can be entirely regenerated from cells, tissues and organs of the plant. Available techniques are reviewed in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

The generation of fertile transgenic plants has been achieved in the cereals rice, maize, wheat, oat, and barley (reviewed in Shimamoto, K. (1994) Current Opinion in Biotechnology 5, 158-162; Vasil, et al. (1992) Bio/Technology 10, 667-674; Vain et al., 1995, Biotechnology Advances 13 (4): 653-671; Vasil, 1996, Nature Biotechnology 14 page 702).

Plants which include a plant cell according to the invention are also provided. Preferred plants include tomato, pepper, aubergine, courgette, lettuce, cabbage, broccoli, ornamentals, potato and yam. Most preferred are lettuce and tomato plants.

In addition to the regenerated plant, the present invention embraces all of the following: a clone of such a plant, seed, selfed or hybrid progeny and descendants (e.g. F1 and F2 descendants). The invention also provides a plant propagule from such plants, that is any part which may be used in reproduction or propagation, sexual or asexual, including cuttings, seed and so on. It also provides any part of these plants, which in all cases include the plant cell heterologous to the gsh1I and gsh2 genes and, optionally, at least one gene involved in the cycling of glutathione between a reduced and an oxidised form as described above.

A plant according to the present invention may be one which does not breed true in one or more properties. Plant varieties may be excluded, particularly registrable plant varieties according to Plant Breeders' Rights.

Plants transformed with vectors of the present invention have been found to have improved root weight and development compared to control plants, enabling improved water and nutrient uptake.

Therefore in a further aspect of the invention, there is provided a method of enhancing root development in a plant comprising the steps of

(i) providing a vector comprising the gsh1 gene, the gsh2 gene under the control of different promoters to enable differential expression of γ-ECS and GS, and optionally at least one gene encoding an enzyme involved in the redox cycling of glutathione between its reduced and its oxidised forms;

(ii) transforming a plant with the vector; and

(iii) allowing replication of the transformed plant.

Furthermore, plants and the fruit of plants transformed with vectors of the present invention have been found to have enhanced glutathione levels at the three ripening stages tested. This suggests that such plants and their fruits will have a longer shelf life.

Therefore in a further aspect of the invention, there is provided a method of enhancing levels of glutathione, and optionally, in particular, of reduced glutathione, in the fruit of a plant comprising the steps of

(i) providing a vector comprising the gsh1 gene, the gsh2 gene under the control of different promoters to enable differential expression of γ-ECS and GS, and optionally at least one gene encoding an enzyme involved in the redox cycling of glutathione between its reduced and its oxidised forms;

(ii) transforming a plant with the vector; and

(iii) allowing replication of the transformed plant.

Moreover, there is also provided a method of increasing the post-harvest shelf life of a plant and/or the fruit of a plant comprising the steps described above.

Plants, for example lettuces, transformed with the vectors of the present invention show that bolting of the plant may be delayed. Therefore the invention further encompasses a method of delaying the bolting of a plant comprising the steps described above.

In addition to use of the vectors of the present invention for enhancing the tolerance of plants to oxidative stress, the information disclosed herein may also be used to reduce the antioxidant activity in cells in which it is desired to do so (thereby alleviating at least some of the effects of oxidative stress tolerance).

The invention will now be further described with reference to the following non-limiting Figures and Examples. Other embodiments of the invention will occur to those skilled in the art in the light of these.

All publications patent applications and references to sequences cited in this specification are herein incorporated by reference as if each individual publication, patent application and sequence were specifically and individually indicated to be incorporated by reference. As described below these include references to: gsh1 (Watanabe et al. (1986) NAR. 14, 4393-4400); gsh2 (Gushima et al. 1984; NAR 12, 9299-9307); gene encoding PHGPX (Mullineaux et al 1998; Plant J. 13, 375-379)[also accession AJ000508]; gene encoding GOR1 (Creissen et al 1992; Plant J. 2, 129-131) [also accession X60373]; cDNA encoding GOR2 (Stevens et al (1997) Plant Mol. Biol. 35 pp641-654) [also accession X98274]; cDNA encoding GST/GPX (Bartling et al 1993; Eur. J. Biochem. 216, 579-586) [also accession X68304]

### FIGURES

FIG. 1 illustrates the AtrpL1-145-atrpL1polyA cassette

FIG. 2 illustrates the pUBQN-apx pA plasmid.

FIG. 3 illustrates the pEF1α-163 plasmid (equivalent to pPIG163)

FIG. 4 illustrates the pE6KL plasmid

FIG. 5 ilustrates the pGreen0049 plasmid

FIG. 6 illustrates the pGSH104 plasmid

FIG. 7 illustrates the pGSH205 plasmid

FIG. 8 illustrates the pGSH3 plasmid.

FIG. 9 illustrates the pE6KL-GSH3 plasmid.

FIG. 10 illustrates the pGPX4 plasmid.

FIG. 11 illustrates the pE6KLGSH3-GPX plasmid

FIG. 12 illustrates the pAtrpL1-GOR1-AtrpL1 polyA

FIG. 13 illustrates the pAFQ70.1 plasmid

FIG. 14 illustrates the pGSH103 plasmid

FIG. 15 illustrates the pGSH204 plasmid

FIG. 16 illustrates pGreen0049GSH4

FIG. 17 illustrates the pAtrpL1-GOR2-AtrpL1 polyA

FIG. 18 illustrates the pUBI-GPX/GST apxpolyA-Bam

FIG. 19 illustrates pGST/GOR2

FIG. 20 illustrates pAFQ70.2

FIG. 21 illustrates foliar glutathione content of transgenic (+) and azygous (−) AFQ70.1 tomato lines

FIG. 22 illustrates glutathione levels in transgenic (70.1#3) and wild-type (wt) at mature green, turning and red ripe stages of fruit development

FIG. 23 illustrates glutathione content of homozygous and azygous progeny of line AFQ70.1.33

FIG. 24 illustrates GSH content of leaves of lettuce transformed with AFQ70.2. Data are for progeny arising from self pollination of primary transformants AFQ70.2.91 and AFQ70.2.126 along with azygous control material from the same seed batch.

FIG. 25 illustrates the response of the transgenic tomato line AFQ70.1.3 to paraquat treatment.

FIG. 26 illustrates tipburn in transgenic (33) plants and azygous controls

FIG. 27 illustrates H₂O₂ levels in a highly expressing AFQ70.1 line and its azygous control at 60 days.

FIG. 28 illustrates lipid peroxidation in the bottom leaves of a high expressing AFQ70.1 line and its azygous control.

FIG. 29 illustrates root fresh weight of one AFQ70.1 line and its azygous control.

### EXAMPLES

1. The Constructs

Two constructs were made for manipulation of expression of enzymes of glutathione metabolism and turnover in the chloroplast (pAFQ70.1) or in the cytosol (pAFQ70.2) of transgenic plants. In addition to the genes and cDNAs directly associated with manipulation of glutathione, a kanamycin resistance gene (nptII) and luciferase gene (luc) were introduced adjacent to the left and right T-DNA border sequences, respectively, to enable selection and easy identification of transgenic plants.

1.1 Construction of the Promoter-PolyA Cassettes

1.1.1 CaMV35S-CaMV PolyA Cassettes

The cassettes incorporating the CaMV35S (with duplicated enhancer region) and CaMV polyadenylation sequences were based on the plasmids pJIT117 (Guerineau et al 1988, Nucl Acids Res. 16, 11380) and pJIT163 (Guerineau et al, 1992; Plant Mol. Biol. 18, 815-818). Modifications to these constructs to facilitate cloning are described in the following sections.

1.1.2 Construction of the AtrpL1 Cassette

The AtrpL1 gene is upstream of, and adjacent to the APX2 gene from Arabidopsis (Santos MCG. PhD thesis, UEA (1995), Santos et al Planta 198, 64-69 (1996)). The sequence forms part of the BAC clone F11F8 (accession number AC016661.5; co-ordinates 51745-52132).

Cloning of AtrpL1 Promoter:

The source of DNA is a 9 kb EcoRI genomic DNA fragment isolated from λAPX5 (15 kb λ GEM clone harbouring both the APX2 gene and the AtrpL1 gene; Santos MCG. PhD thesis, UEA (1995)) cloned into pBluescriptSK+, orientated such that the 5′ end is at the KpnI side of the polylinker (designated 5E2). From this a ca. 500 bp fragment was isolated using the restriction sites KpnI (in the polylinker) to XmnI (GAANN′NNTTC; co-ord 462 in the atrpL1 sequence; Santos 1995, wherein N is A or G or C or T). This was done by cutting with XmnI followed by T4 polymerase treatment and then digesting with KpnI. The resulting fragment was cloned into KpnI-HincII sites of pBluescriptSK+ as an intermediate clone. The KpnI-HindIII fragment from this intermediate was isolated and inserted into KpnI-HindIII sites of pJIT145. The cassette pJIT145 is equivalent to pJIT163 except that the SacI site at the 5′ end of the enhanced CaMV 35S promoter is replaced by a BglII site. Thus the AtrpL1 promoter replaces the 35S promoter sequences in pJIT145 (designated AtrpL1/145)

Cloning of AtrpL1 PolyA Sequence

5E2 DNA (see above) was digested with RsaI (co-ord 2070) and EcoRV (co-ord 2330). The 160 bp atrpL1 polyA fragment was eluted from a polyacrylamide gel. This fragment was ligated into the EcoRV-SmaI sites of pJIT145 to create pJIT145/rpL1polyA.

Cassette Assembly

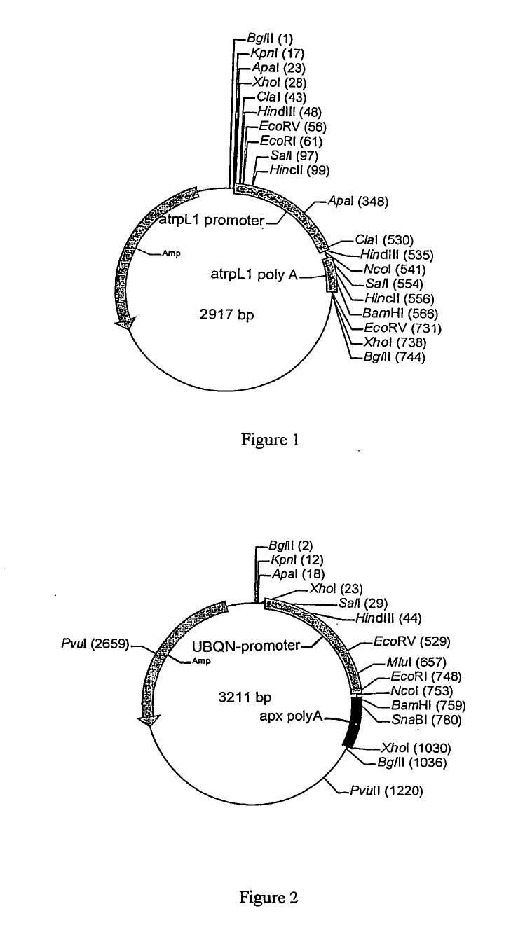

The CaMV 35S promoter sequences in pJIT145/rpl1polyA were replaced with the Atrp1 promoter from AtrpL1/145 as KpnI-NcoI fragment into the same sites to create AtrpL1-145-atrpL1polyA, which is shown in FIG. 1.

1.1.3. Construction of the UBQN-Apx1polyA Cassette.

The fragment carrying the UBQ1 gene promoter region (Collin et al, 1990, J. Biol. Chem 265 12486-12493) is in a plasmid called p1933 and is a HindIII-BglII fragment cloned into the HindIII-BamHI sites of pBI101.3 (from Clontech).

Subcloning of the UBQ Promoter Region.

The ca. 750 bp SmaI fragment from p1933 was cloned into the EcoRV site of pNondescript (Edwards et al (1996), Plant Phsiol. 112 pp89-97; Creissen et al 1999) and the orientation of the fragment in the plasmid chosen such that the 3′ end of of the promoter fragment was adjacent to the NcoI site in the pNondescript plasmid. This plasmid was called pUBQNS-12.

The APX1 PolyA Cassette.

The source of APX1 polyA fragment was clone 18AE (a ca.50.1 kb ApaI-EcoRI subclone from APX18 in pBluescript SKII+; Santos 1995). The polyA sequences are located on a 263 bp RsaI fragment (coordinates 2340-2603) based on the sequence data from Kubo et al 1993 (FEBS 315, 313-317; accession number X70220)

This fragment was cloned into the SmaI and EcoRV sites of pJIT145, replacing the CaMV polyA. The orientation of the fragment was checked by SnaB1 and BglII double digests, followed by sequencing. This plasmid was designated pJIT145-apx polyA.

Assembly of the UBQ-Apx PolyA Cassette

A pUBQ-apx polyA cassette was constructed by recovering a 750 bp Asp718-BamHI fragment carrying the UBQ1 promoter from pUBQNS-12 and inserting into the same sites-of pJIT145-apx polyA, thus replacing the CAMV 35S promoter in pJIT145-apx polyA with the UBQ1 promoter. This made plasmid pUBQ-apx polyA.

It was subsequently discovered on sequencing the insert in PUBQNS-12, that in fact some of the UBQ1 5′ end cds was present, including an ATG which could interfere with translation intitiation from the intended ATG (as part of the NcoI site). The ATG was removed by substituting a synthetic DNA fragment of the same sequence but carrying ACG instead (bold, italic and underlined). There is a MluI site in the UBQ1 promoter upstream of the 5′ transcription start site (/->) which was the 5′ end of the fragment. The 3′ end of the fragment contained a BamHI compatible end (underlined).

```
    MluI
                                          /→ UBQ1 transcript initiation
ACGCGTACATTGACATATATAAACCCGCCTCCTCCTTGTTTAGGGTTTCTACGTGAGAGAAGACGAAACACCCAAGACGCAGATCCCCATCGAATTCGATC
                                                                                                                                                                                                EcoRI
```

This fragment was cloned into pUBQ-apx polyA MluI and BamHI sites and after ligation the DNA mixture was backcut with BamHI to select the modified plasmids. This plasmid was called pUBQ-apx polyA Bam0. The final plasmid, pUBQNapx pA was built by swapping the unmodified promoter in pUBQ-apx polyA for the modified one from pUBQ-apx polyA Bam0. This last step restored a BamHI site and NcoI site downstream of the modified promoter. This plasmid was called pUBQN-apx pA and is shown in FIG. 2.

1.1.4 EF1α-CaMV PolyA Cassette

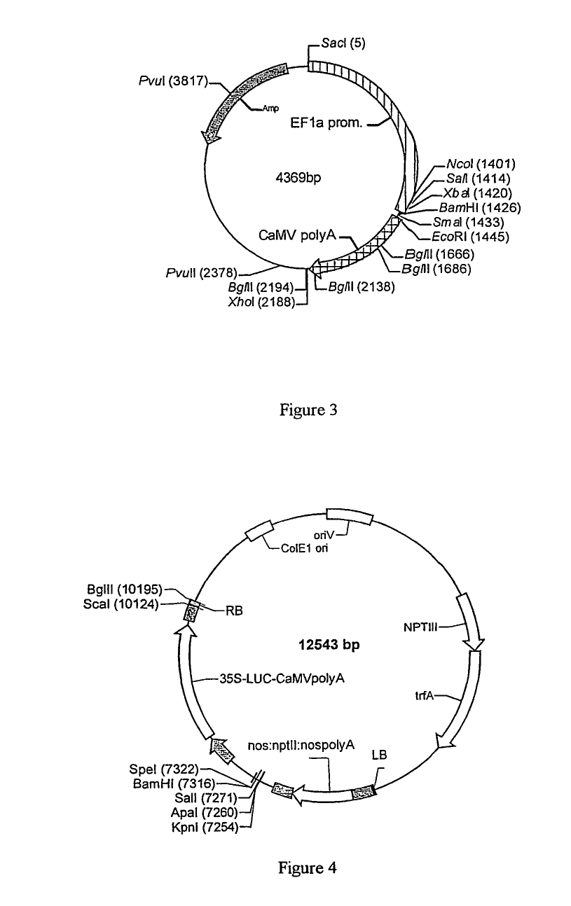

The Ef1α promoter (Liboz et al. Plant Mol. Biol. 14:107-110(1989) equivalent to BAC clone T6D22, accession number AC026875, co-ordinates 10054-11457)) was recovered from plasmid pCCpIGUS (Axelos et al, MGG 219: (1-2) 106-112 (1989)) as a 1.4 kb Sac I-NcoI fragment and inserted into the same sites of pJIT163 replacing the enhanced CaMV 35S promoter with the EF1α promoter. This plasmid is pPIG163, also now called pEF1α-163 and is shown in FIG. 3.

1.2 Binary Vectors

1.2.1 Construction of E6KL.

For AFQ70.1 the vector E6KL was used. This consists of the plasmid backbone of pBIN19 (minus its T-DNA) (Bevan 1984; NAR 12, 8711-8721) with the synthetic 685 bp T-DNA which is now also part of pGreen0000 (Hellens et al 2000; Plant Mol Biol 42, 819-832; www.pgreen.ac.uk). This plasmid was made by inserting the T-DNA as a 715 bp BglII fragment into the unique BglII site of pRK2-BglII (Bevan 1984 (ibid); Jones et al 1998; J. Gen Virol 79, 3129-3137). This basic binary Ti plasmid was called pE6. A nos:nptII:nos cassette for kanamycin selection in transformed plants (Hellens et al 2000; ibid) was inserted as an EcoRV fragment in to the HpaI site just internal to the T-DNA left border, creating pE6Kan. A 35S-LUC+-CaMV polyA cassette (Hellens et al 2000; ibid) was inserted as an EcoRV fragment into the unique StuI site, adjacent and internal to the RB, to create pE6KL as shown in FIG. 4. Thus, genes to be transferred are sandwiched between the nptII and LUC border markers, using the lacZ′-multiple cloning polylinker present in the plasmid

1.2.2 pGreen0049

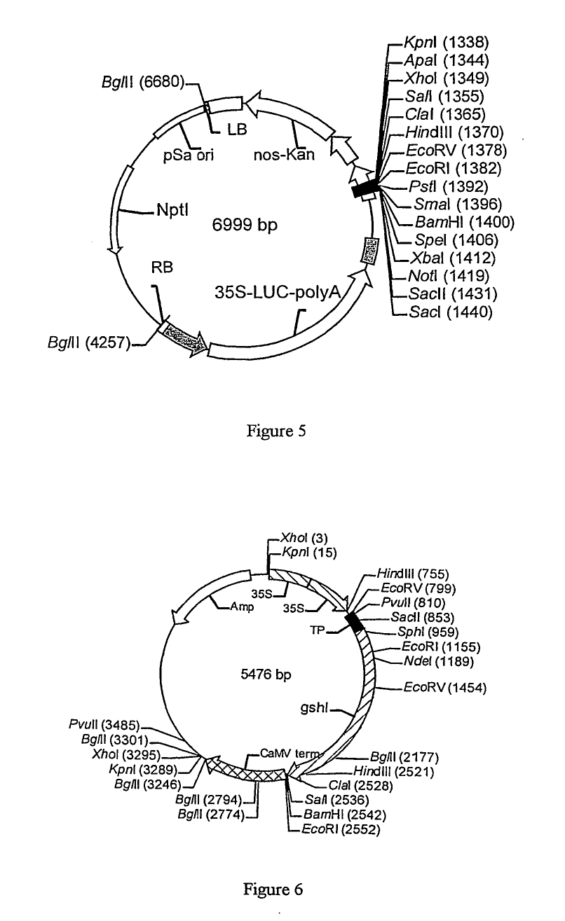

For AFQ70.2 the pGreen vector pGreen 0049 (as shown in FIG. 5) was used instead of E6KL. Full details of this vector are available in the pGreen website (www.pgreen.ac.uk).

2. AFQ70.1

2.1 cpGSHI/II.

2.1.1 PCR amplification and cloning of E. coli gsh1 and gsh2 genes. Genes encoding GSHI and GSHII were cloned from E. coli B DNA (Creissen et al 1999; Plant Cell 11, 1277-1291). Primers were designed in order to amplify the gsh1 and gsh2 genes. For gsh1 (Watanabe et al. (1986) NAR. 14, 4393-4400) these were: forward primer 1057 5′-CATGATGTGGTGGCACTAATTGTAC-3′ (5′ co-ord=247; Acc no. X03954) and reverse primer 1058 5′-CTGTCAGGCGTGTTTTTCCAGCCAC-31 (51 co-ord=1897; Acc no. X03954). For gsh2 (Gushima et al. 1984; NAR 12, 9299-9307) these were forward primer A1059 5′-TGATTGGCCCGGAAGGCGGTTTATC-3′ (5′ co-ord=196; Acc no. X01666) and reverse primer A1060 5′-TCAGAGTCTCAACGAGATCCTTCTC-3′ (5′ co-ord=1352; Acc no. X01666). 1 pg E. coli B DNA (Sigma) was used in PCR reaction with each pair of primers, PCR conditions were: 30 sec @92° C.; 30 sec @55° C.; 1 min 30 sec @72° C.; 40 cycles, 20 min @ 72° C. final extension. The gsh1 (1.65 kb) and gsh2 (1.15 kb) fragments were eluted from agarose gels and ligated into EcoRV digested, ddTTP-tailed pBluescript KSII+ to generate pGSH101 (gsh1) and pGSH201 (gsh2). Each of these constructs was tested for function by complementation of E. coli mutants gshA821 (deficient in gsh1) and gshB830 deficient in gsh2) restoring ability to to synthesise glutathione and to grow on minimal medium containing tetramethyl thiuram disulphide (TMTD), to which glutathione deficient mutants are highly sensitive.

2.1.2. Site Directed Mutagenesis (SDM).

For SDM, the gshI and gshII genes were subcloned into pAlter using the BamHI and SalI sites in pAlter and in pGSH101/pGSH201 to create pAlter/gshI and pAlter/gshII respectively. Mutagenesis was performed on single stranded DNA using the primers A315 5′-GGGAGGTCAGCATGCTCCCGGACGTATC (pAlter/gshI) and A315 5′-CGGGAGGTCAGCATGCTCCCGGACGTATC (pAlter/gshII) to introduce the required SphI site (bold) at the translation initiation site (underlined) and the amp repair oligo supplied with the kit. Mutated plasmids were recovered in BMH 71-18 mutS, in liquid culture (plus ampicillin) and a miniprep from this was tested for function by complementation of the gshA821 and gsh830 mutants of E. coli, restoring ability to grow on minimal medium containing tetramethyl thiuram disulphide (TMTD).

The modified constructs containing the introduced SphI site at the AUG start codon (gcATGc) are called pGSH1-S and PGSHII-S.

2.1.3 Subcloning Under Control of CaMV 35S Promoter and Polyadenylation Sequences

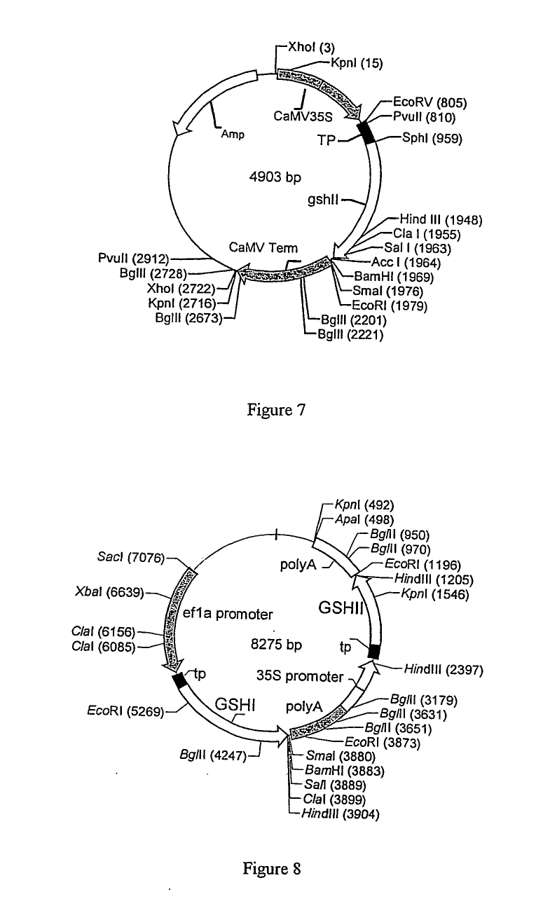

The modified gshI and gshII genes were subcloned into the vector pJIT260 (pJIT260 is identical to pJIT117 (Guerineau et al 1988; NAR 16, 11380) except that the SacI site 5′ to the CaMV promoter sequences has been replaced with an XhoI site) using the SphI and SalI sites in pJIT260 and pGSH1-S/pGSHII-S to create pGSH104 as shown in FIG. 6 and pGSH205 as shown in FIG. 7 respectively.

2.2 Assembly of EF1α-cpGSHI-PolyA/CaMV35S-CpGSHII-PolyA Cassette (pGSH3)

Modifications to pGSH104

A PCR product from pGSH104, consisting of the transit peptide and part of the GSHI coding sequenc was obtained using oligos A3332 (5′-GAAGTGAGAACCATGGCTTCTATG-3′) and A3333 (5′-CGCGCATAAAGGTCAGCATATG-3′). This PCR inserted a NcoI site at the ATG of the transit peptide. The PCR fragment was cut with NcoI and EcoRI and cloned into the same sites of pNondescript. This created PNS-TP. Then a SphI-SalI fragment (mature coding sequence) from pGSH104 was inserted into same sites of PNS-TP, thus creating a TP-GSHI coding sequence with a NcoI site at the ATG of the TP in plasmid PNS-TPGSHI.

Modifications to pGSH205

pGSH205 was cut with EcoRI and ClaI (T4 poll treated and religated. This removed sites at 3′ end of the polylinker. The resulting plasmid was cut with BglII and religated, deleting 500 bp of CaMV poly A (not required) and leaving a unique XhoI site at 5′end of CaMV 35S promoter, creating pGSH205del. This plasmid was cut with XhoI, T4 poll treated and a BamHI linker inserted. This plasmid is called pGSH205del-Bam.

Insertion of 35S:tpGSHII-polyA 3′ to EF1α Promoter Cassette in pEF1α-163.

pEF1α-163 was cut with BglII in CaMV polyA and the 35S:tpGSHII-polyA was inserted into this site as a BamHI-BglII fragment recovered from pGSH205del-Bam creating pPIGGSH205. The BglII site at the extreme end of the CaMV polyA attached to the TPGSHII gene was cut, T4 polI treated and an ApaI linker (GGGCCC) inserted, thus introducing a unique ApaI site into the plasmid, now called pPIGGSH205-Apa.

This plasmid was digested with NcoI and SalI. The tp-GSHI coding sequence was recovered from PNS-TPGSHI as an NcoI-SalI fragment and inserted into the same sites in pPIGGSH205-Apa. Thus EF1α-tpGSHI-CaMVpolyA and 35S-tpSHII-CaMVpolyA are in tandem. This is called pGSH3 and is shown in FIG. 8.

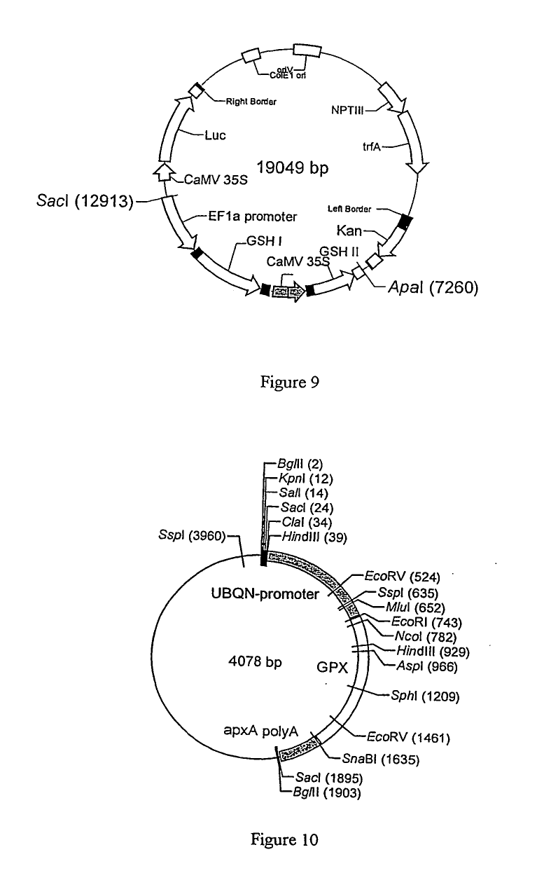

The EF1α-TPGSHI CaMV polyA and 35S-TPGSHII CaMV polyA genes were recovered as an SacI-ApaI fragment and inserted into the same sites of the binary Ti vector, pE6KL, creating pE6KL-GSH3 as shown in FIG. 9.

2.3 Construction of Ubi (UBQ)-GPX-ApxpolyA.

An 868 bp EcoRI-SspI PHGPX coding sequence fragment was recovered from pGPX2. This plasmid contains a full length coding sequence for pea plastidial phospholipidhydroperoxide glutathione peroxidase (PHGPX; Mullineaux et al 1998; Plant J. 13, 375-379). This was inserted into the BamHI (rendered blunt ended by T4 poli treatment)-EcoRI sites of pUBQN-apx pA, creating pGPX4 (FIG. 10).

A synthetic DNA fragment was made by annealing the following 2 oligonucleotides together; 5′-ACCGTCGACGAGCTCGTACGGTATCGA-3′ and 5′-TCGATCGATACCGTACGAGCTCGTCGACG-3′. which would replace the order of restriction sites in the 5′ end of the UBQ promoter from BglII, Asp718, ApaI, XhoI, SalI, HindIII to BglII, Asp718, SalI, SacI, ClaI, HindIII. This was achieved by ligating the synthetic fragment into the Asp718 and SalI sites of pGPX4 and cutting with ApaI after ligation. This created pGPX4-Sac1. pGPX4-Sac1 was cut with XhoI (3′end of APX1 polyA) and a SacI adaptor oligonucleotide (5′-TCGACGAGCTC-3′) was ligated into the site, destroying the XhoI site and adding in a SacI site to create pGPX4-Sac2.

2.4 Insertion of UBQN-GPX-ApxpA into pE6KL-GSH3.

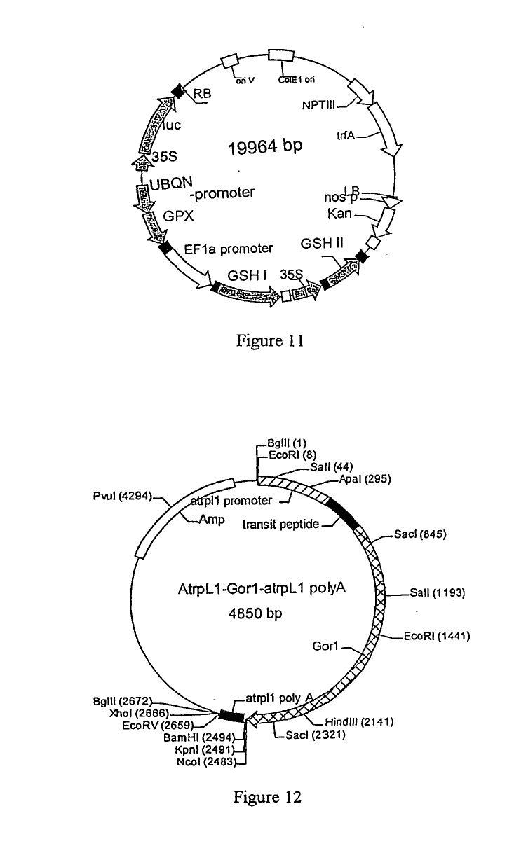

The 1.85 kb UBQN-GPX-apxpA from pGPX4-Sac2 was inserted as a SacI fragment into the unique SacI site of pE6KLGSH3 and the orientation of the GPX gene selected to be driving transcription in the same direction as GSHI and GSHII. This plasmid is called pE6KLGSH3-GPX (FIG. 11). The GPX gene is inserted between the RB 35S-LUC marker gene and the cpGSHI gene.

2.5 Assembly of AtrpL1-Gor1-AtrpL1 PolyA

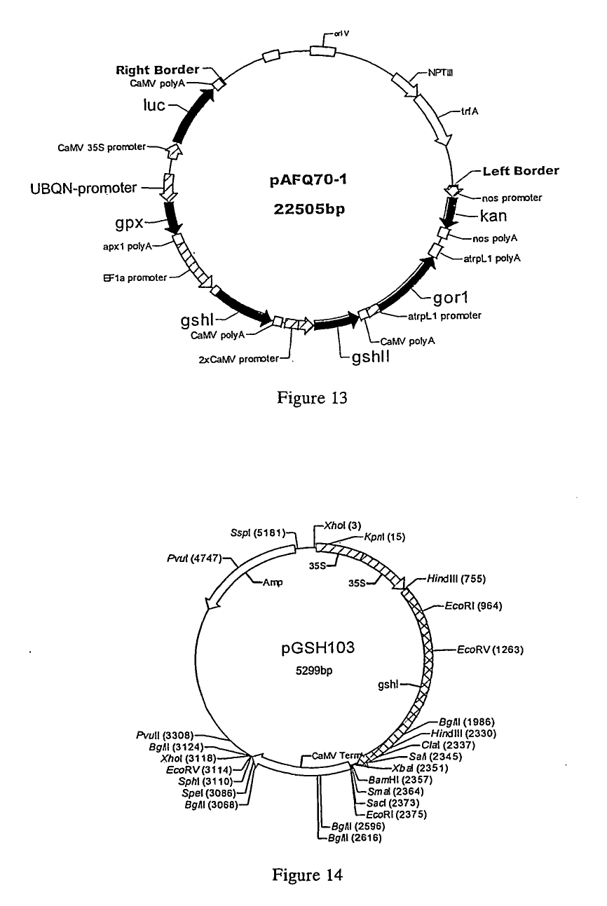

Part of the polylinker was deleted from atrpL1-145-atrpL1 polya (FIG. 1) by treatment with KpnI/T4 polymerase; EcoRV and religation (to create atrpL1D). Then the GOR1 cDNA was isolated as an EcoRV-BamHI fragment from pGR202 (containing the full length GR201 cDNA sequence (Creissen et al 1992; Plant J. 2, 129-131). This fragment was ligated into the ClaI/T4 poll treated-BamHI sites of atrpL1D to create AtrpL1-Gor1-atrpL1 polyA (FIG. 12). A PvuI site was introduced at the 3′ end of the atrpL1 polyA (replacing XhoI) to create atrpL1-gor1-PvuI. This was digested with ApaI (in AtrpL1 promoter) and PvuI and the eluted ca. 2.4 kb fragment was inserted into the unique Apa1/PvuI sites in E6KLGSH3GPX. Finally the missing 5′end of the AtrpL1 promoter was restored as a Apa1 fragment from AtrpL1-145-atrpL1 polyA into the unique Apa1 site in E6KLGSH3GPX, the orientation checked by XhoI digestion, to create pAFQ70.1 (FIG. 13).

3. AFQ70.2

3.1 Cytosolic GSHI/II.

The PCR amplification of gshI and gshII has been described elsewhere (Creissen et al 1999). The PCR products from gshI and gshII were cloned into EcoRV digested, ddTTP-tailed pBluescript KSII+to generate pGSH101 and pGSH201 respectively. These plasmids were tested for function by complementation of the E. coli gshI mutant, gshA821, or the gshII mutant, gshB830, restoring their ability to grow on minimal medium containing tetramethyl thiuram disulphide (TMTD)

3.1.1 GSHI Manipulation

The gshI gene was subcloned into pAlter (Promega) using the BamHI and SalI sites in pAlter and in pGSH101 the resulting plasmid was called pAlter/gshI. Mutagenesis was performed on single stranded DNA according to the manufacterer's instructions using the primer A1075 5′-CGGGAGGTCACCATGGTCCCGGACGTATC to introduce the required NcoI site (bold) at the translation inition site (underlined) and the amp repair oligo supplied with the kit.

Mutated plasmids were recovered in BMH 71-18 mutS, in liquid culture (plus ampicillin) and a miniprep from this was used to transform DH5α. The introduction of the NcoI site was confirmed and the new construct was tested for function by transforming the E. coli gshI mutant strain, gshA821, restoring its ability to grow on minimal medium containing tetramethyl thiuram disulphide (TMTD). The modified construct containing the introduced NcoI site at the AUG start codon (ccATGg) is called pGSH1-N

Subcloning Under Control of CAMV 35S Promoter and Polyadenylation Sequences

The modified gshI gene was subcloned into the vector pJIT169 (pJIT169 is identical to pJIT163 except that the SacI site 5′ to the CaMV promoter sequences has been replaced with an XhoI site) using the NcoI and SalI sites in pJIT169 and pGSH1-N to create pGSH103 (FIG. 14)

3.1.2 GSHII Manipulation

The gshII gene was subcloned into pAlter using the BamHI and SalI sites in pAlter and in pGSH201 this was called pAlter/gshII. Mutagenesis was performed on single stranded DNA according to the manufacterer's instructions using the primer A314 5′-CGGAGAAGAACCATGGTCAAGCTCGGC-3′ to introduce the required NcoI site (bold) at the translation inition site (underlined) and the amp repair oligo supplied with the kit. Mutated plasmids were recovered in BMH 71-18 mutS, in liquid culture (plus ampicillin) and a miniprep from this was used to transform DH5α. Checked by restriction analysis (for introduction of NcoI site) and by transforming gshB830, restoring ability to grow on minimal medium containing tetramethyl thiuram disulphide (TMTD). The modified construct containing the introduced NcoI site at the AUG start codon (ccATGg) is called pGSHII-N

Subcloning Under Control of CaMV 35S Promoter and Polyadenylation Sequences

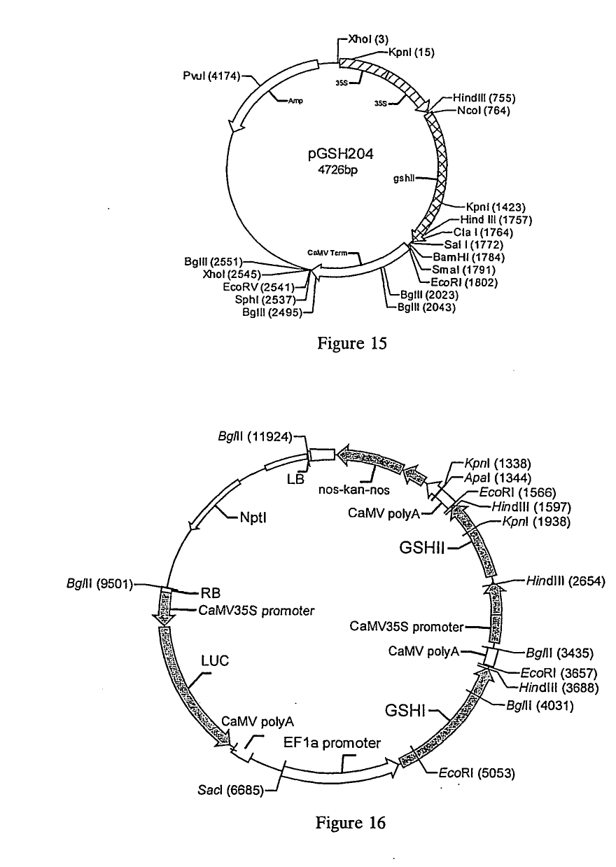

The modified gshII gene was subcloned into the vector pJIT169 (pJIT169 is identical to pJIT163 except that the SacI site 5′ to the CaMV promoter sequences has been replaced with an XhoI site) using the NcoI and SalI sites in pJIT169 and PGSHII-N to create pGSH204 (FIG. 15)

3.1.3 Assembly of EF1a-Gsh1-Ef1a PolyA/CaMV32S-GshII-PolyA Plasmid.

Modifications to pGSH204

pGSH204 was cut with EcoRI and ClaI, T4 polI treated and religated to remove sites at at 3′ end of polylinker. Then this plasmid was cut with BglII and religated. This deletes 500 bp of CaMV poly A (not required) and leaves a unique XhoI site at the 3′end of CaMV polyA, creating pGSH204del. This plasmid was cut with XhoI, T4 polI treated and a BamHI linker inserted. This plasmid is called pGSH204del-Bam.

Insertion of 35S: GSHIIgene 3′ to EF1a Promoter Cassette.

pEF1α-163 (Arabidopsis EF1a promoter:CaMV polyA) was cut with BglII in CaMV polyA and the 35S:GSHII gene was inserted into this site as a BamHI-BglII fragment recovered from pGSH204del-Bam, creating pPIGGSH204. The BglII site at the extreme end of the CaMV polyA attached to the GSHII gene was cut, T4 polI treated and an ApaI linker (GGGCCC) inserted, thus introducing an unique ApaI site into the plasmid, now called pPIGGSH204-Apa.

Insertion of GSHI into pPIGGSH204-Apa.

pBluescript SKII+ was cut with SalI, T4 polI treated and re-ligated to destroy the SalI site and create pBluescript-Sal del. The EF1α promoter from pPIG163 was subcloned in to pBluescript-Sal del, as a SacI-BamHI fragment into the same sites, creating pBS-EF1α. The GSHI gene from pGSH103 was inserted as a NcoI-SalI fragment into pBS-EF1α, thus making an EF1α-GSHI fusion. The EF1α-GSHI was inserted as a SacI-BamHI fragment into the same sites of pPIGGSH204-Apa, replacing the EF1α promoter with an EF1α-GSHI fusion. This plasmid was called pGSH4

3.1.4. Assembly of GshI/GshII Genes into pGreen0049

The GSHI/GSHII fragment from pGSH4 was inserted as an ApaI-SacI fragment into the same sites of pGreen0049 (Hellens et al 2000, ibid). This plasmid was called pGreen0049GSH4 (FIG. 16).

3.1.5. Construction of AtrpL1-GOR2 Fusion.

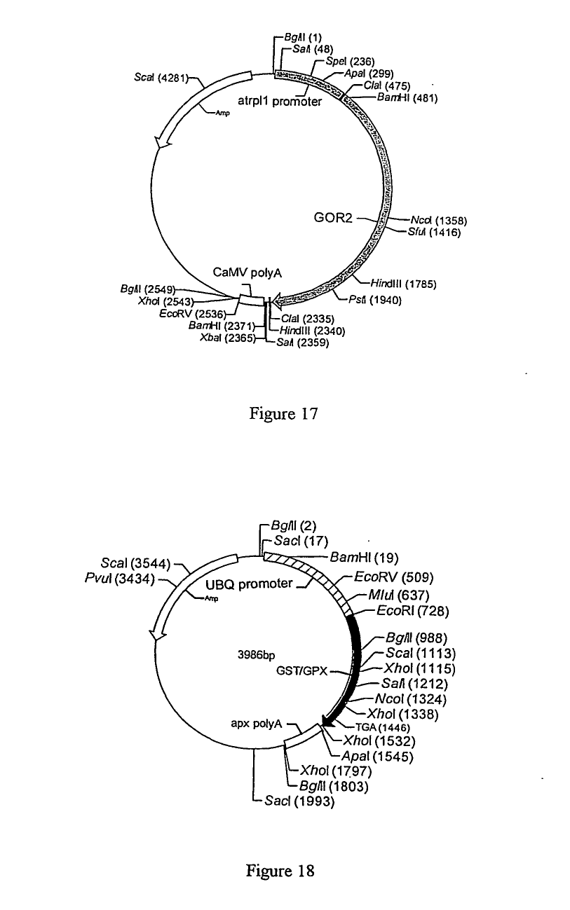

The pea cytosolic GR cDNA in pBluescriptKSII (KSII-GOR2;Stevens et al (1997) Plant Mol. Biol. 35 pp641-654) was cut with SpeI, T4 polI treated and a ClaI linker (GCATCGATGC) was inserted to create pKSII-GOR2-ClaI. At the 3′end of the plasmid the multiple restriction sites were removed by cutting with KpnI (2 sites at 1890 and 1963), T4-polI treated and inserting the same ClaI linker. This replaced a mass of restriction sites with a ClaI site at the 3′end of the GOR2 cDNA. This plasmid was called pKSGOR2 kpndelCla. The GOR2 cDNA could now be recovered as a 1.84 kb ClaI fragment and inserted into the unique ClaI site of the pAtrpL1-AtrpL1polyA cassette plasmid. The correct sense orientation, with respect to the AtrpL1 promoter was selected and this plasmid was called pAtrpL1-GOR2-AtrpL1 polyA (FIG. 17).

3.1.6 Construction of UBQ-GST/GPX-Apx PolyA

The GST/GPX cDNA was recovered from a pBluescript vector plasmid (Bartling et al 1993; Eur. J. Biochem. 216, 579-586) as KpnI-T4 polI treated-EcoR1 fragment of 1 kb. This fragment was inserted into the SnaBI-EcoRI sites of pUBQ-APXpolyA, to create pubi-GPX/GST.

A SacI linker (GGAGCTCC) was inserted into the unique PvuII site, 3′ to the APXpolyA at coordinate 2007. Then at the 5′ end of the promoter the Asp718 (coordinate 8) to HindIII (coordinate 44) in pUBI-GPX was replaced with a Asp718-HindIII adaptor (destoys both sites upon insertion) carrying SacI and BamHI site sites (5′ to 3′). This plasmid was called pUBI-GPX/GST apxpolyA-Bam (FIG. 18).

3.1.7 Construction of a Combined GOR2 and GST/GPX Plasmid.

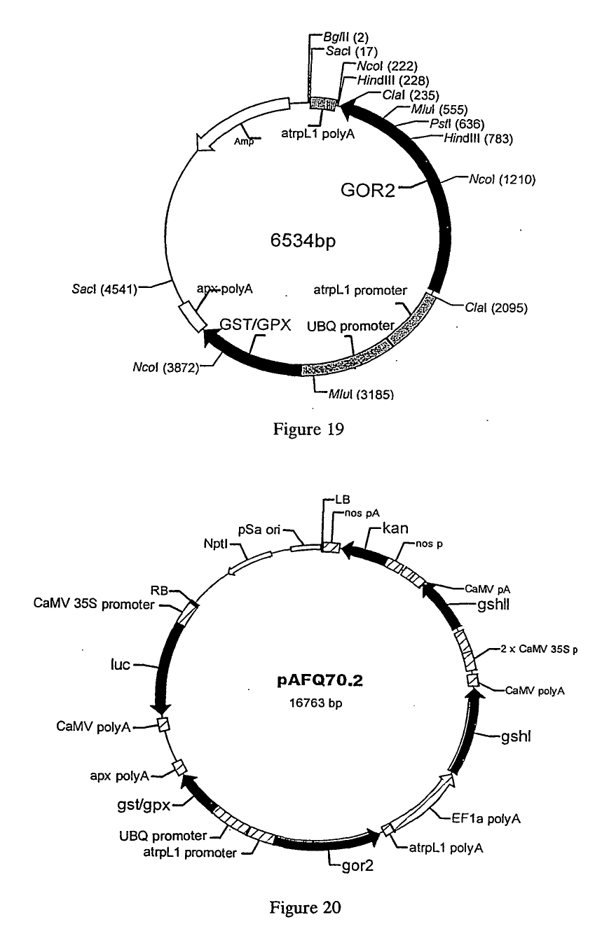

The pAtrpL1-GOR2-AtrpL1 polyA plasmid DNA was cut with BglII and the atrpL1-gor2-atrpl1 poly fragment (2.Gkb) was eluted from the agarose gel and inserted into the BamHI site (coordinate 19) of pUBI-GST/GPX. To create pGST/GOR2 (FIG. 19). The orientation which gave each gene transcribing away from each other was the only orientation recovered (see map).

3.2 Assembly of pAFQ70.2

The 4.5 kb GST/GOR2 band from pGST/GOR2 was recovered as a SacI fragment and inserted into the unique SacI site of pGreen0049GSH4, creating pAFQ70.2 (FIG. 20).

4. Agrobacterium Mediated Transformation

The plasmids AFQ70.1 and AFQ70.2 were electroporated into the Agrobacterium strain AGL1 (in the case of the pGreen0049 based AFQ70.2, the AGL1 strain also harboured the pSOUP plasmid required for pGreen replication; Hellens et al, 2000, ibid). These strains were then used for plant transformation.

4.1 Tomato Transformation

4.1.1. Plant Material

Seeds of tomato, Lycopersicum esculentum (var FM6203) were surface sterilised for 2 hours in a solution of 10% sodium hypochlorite; rinsed 3 times in sterile distilled water and planted in a magenta pot containing 50 mls MS basal media supplemented with 3% sucrose and 0.9% agar. The seedlings were grown at 26° C., in a culture room at 3000 lux 16 hour day/8 hour night for 7 days

4.1.2 Agrobacterium-Mediated Transformation

Agrobacterium tumefaciens strain AGL1 containing AFQ70.1 or AFQ70.2:pSOUP was grown overnight in Lennox broth (5 g/L Na Cl, Yeast Extract 10 g/L, Bacto tryptone 10 g/L) at 28° C. on a shaker. The culture was centrifuged at 3000 rpm for 10 minutes and the cell pellet was resuspended in liquid MS basal media supplemented with 3% sucrose. To inoculate the plant material, the cotyledons were removed from seedlings, cut across tip of cotyledon and again across the midrib of the tissue and the cotyledon pieces were incubated with the Agrobacterium suspension for 10 minutes. The explants were then removed from the culture and blotted dry on sterile filter paper. Explants were plated face down onto sterile Whatman filter paper (no. 7) on top of a nurse culture prepared by adding 2 mls of Nicotinia benthaminiana cell suspension to a plate containing 25 mls of MS salts BS vitamins (1 mg/L Nicotinic acid, 1 mg/L pyridoxine, 10 mg/L thiamine, 100 mg/L inositol) supplemented with 1 mg/L 2,4-D, 2 mg/L BAP and 0.8% agar. The cotyledon explants were incubated under low light (2000 lux) at 26° C. After 2 days the explants were removed from the nurse plates and plated face upwards onto selection media (MS basal media supplemented with 2% sucrose, Nitsch vitamins, 10 mg/L inositol, 5 g/L agargel, cefotaxime 500 mg/L and kanamycin 10 mg/L). The explants were incubated at 26° C., 16 hour day/8 hour night in a light intensity of 3000 lux and transfered to fresh selection medium every 2 weeks. Shoots appearing on the cut edges of the explant were transferred onto rooting selection media (MS basal media supplemented with 3% sucrose, 0.9% agar, cefotaxime 500 mg/L and kanamycin 100 mg/L). Rooted shoots were subsequently potted on and grown up for further analysis and seed production.

4.2. Lettuce Transformation

4.2.1 Plant Material

Lettuce seeds (Lactuca sativa L. cv. Evola) were supplied by Leen de Mos ('s-Gravenzande, P.O. Box 54-2690 AB, The Netherlands). Seeds were surface sterilised by immersion in 10% (v/v) ‘Domestos’ bleach (Lever Industrial, Runcorn, UK) for 30 min, followed by 3 washes in sterile distilled water. The seeds were placed on agar-solidified (0.8% w/v) half-strength Murashige and Skoog medium with 1.0% (w/v) sucrose, at pHS.8 (20 ml aliquots/9 cm Petri dish; 30-40 seeds/dish). Seeds were germinated at 23±2° C. (16 h photoperiod, 350 μmol m⁻² s⁻¹, Daylight fluorescent tubes). Cotyledons and first true leaves were excised after 7 d and 9 d, respectively, for bacterial inoculation.

4.2.2 Bacterial Strains and Plasmids

Bacteria were grown from −70° C. glycerol stocks at 28° C. on Luria broth (LB) (Sambrook et al., 1989) semi-solidified with 1.5% (w/v) agar and supplemented with the appropriate antibiotics. Overnight liquid cultures were incubated at 28° C. on a horizontal rotary shaker (180 rpm) and were initiated by inoculating 20 ml of liquid LB medium, containing the appropriate antibiotics, in 100 cm³ conical flasks. Bacterial cultures were grown to an O.D.₆₀₀ of 1.0-1.5 prior to inoculation of explants.

4.2.3 Plant Transformation and Growth Conditions

Cotyledons and first true leaves excised from 7 d-old and 9-day old seedlings, respectively, were inoculated with A. tumefaciens and transgenic shoots regenerated using an established procedure (Curtis et al (1994) J. Exp. Bot. 45 pp1441-1449). Shoots which regenerated from explants on medium containing kanamycin sulphate (50 mg L⁻¹) were rooted in vitro in the presence of kanamycin sulphate (50 mg L⁻¹) before transfer to the glasshouse, where they were allowed to self-pollinate and to set seed. Seeds were collected and stored at 4° C. Seeds were sown on the surface of moist peat based compost (Levingtons M3) in 9 cm diameter plastic pots. The pots were placed in an incubator and the seeds were germinated in a growth room at 19° C. with a 16 h photoperiod (350 μmol m⁻² s⁻¹). At 7 days post-sowing (dps) individual seedlings were transferred to 4 cm×4 cm×5 cm peat blocks and kept under the same conditions. At 30 dps, individual plantlets were transferred to 9 cm plastic pots containing a compost mix of John Innes No.3: Levingtons M3: Perlite 3:3:1. The pots were placed individually in 12 cm diameter plastic trays containing 5-10 mm of tap water which was replaced every 24 h. Pots were spaced 10 cm apart to prevent shading from adjacent plants and kept under the conditions described above.

5. Results—Tomato

5.1 AFQ70.1 Lines

5.1.1 Expression of Transgenes.

Analysis of expression of the gshI, gshII, gor1 and gpx transgenes was performed by a combination of RNA gel blot hybridisation and 3′RACE PCR. Two lines which showed expression of all four genes at all of the stages analysed were identified for further analysis (Table 1)

TABLE 1
Transgene expression in selected AFQ70.1 lines of tomato

| | RNA | | | | |
|---|---|---|---|---|---|
| Line | | Mature | | | |
| n° | | green | Turning | Red ripe | DNA |
| #3 | gsh1: + | gsh1: + | gsh1: + | gsh1: + | Single insertion, |
| | gsh2: + | gsh2: + | gsh2: + | gsh2: + | complete |
| | gpx: + | gpx: + | gpx: + | gpx: + | LUC probe: single |
| | gor1: + | gor1: + | gor1: + | gor1: ? | fragment border |
| | | | | | gsh1 probe: |
| | | | | | correct size |
| | | | | | fragment |
| #75 | gsh1: + | gsh1: + | gsh1: + | gsh1: + | Single insertion, |
| | gsh2: + | gsh2: + | gsh2: + | gsh2: + | complete |
| | gpx: + | gpx: + | gpx: + | gpx: + | LUC probe: single |
| | gor1: + | gor1: + | gor1: + | gor1: ? | fragment border |
| | | | | | gsh1 probe: |
| | | | | | correct size |
| | | | | | fragment |

5.1.2 Glutathione Content of Transgenic (AFQ70.1) Tomato Leaves

Glutathione content was measured in leaves and fruit of transgenic lines expressing the introduced transgenes carried on the AFQ70.1 T-DNA. Glutathione was determined by derivatisation with monobromobimane (MB; Newton et al 1981; Anal Biochem. 114, 383-387) and detection of the MB-derivatized products by HPLC as previously described (Creissen et al 1999, ibid). Comparisons were made with control plants that did not contain the transgenes, but were derived from the same, self-pollinated parent (azygous controls)

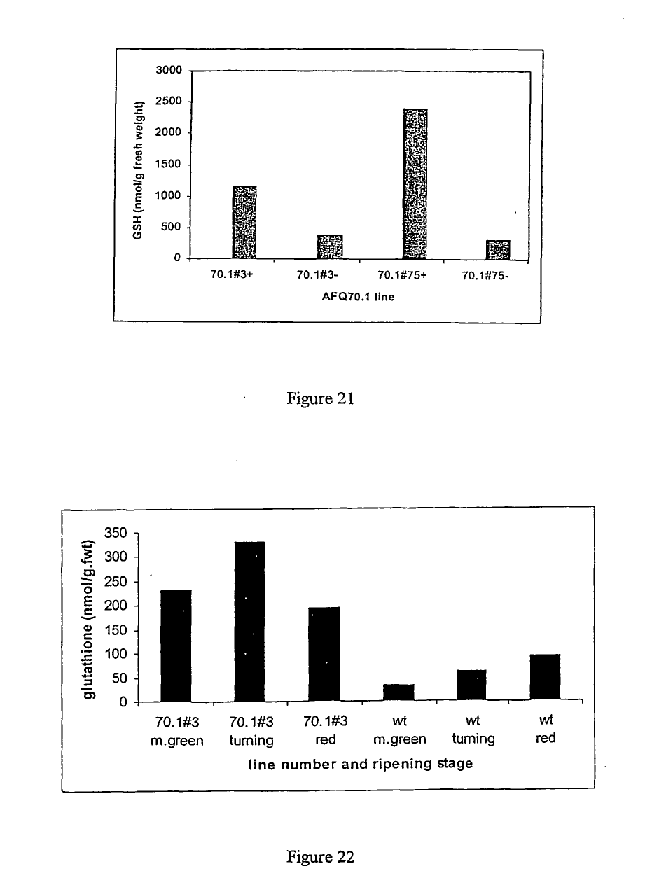

Foliar glutathione content of transgenic (+) and azygous (−) AFQ70.1 tomato lines is illustrated in FIG. 21.

5.1.3 Glutathione Content of Transgenic (AFQ70.1) Tomato Fruits

Glutathione levels were measured at three ripening stage in transgenic fruit and in wild-type controls. The glutathione content at each of the stages was significantly higher than the control level. An example for line AFQ70.1#3 is given in FIG. 22.

5.2 AFQ70.2

5.2.1 Expression of Transgenes

Expression of the gsh1, gsh2, gst/gpx and gor2 transgenes was confirmed by a combination of Northern blotting and 3′ RACE PCR analysis. In addition the complexity of T-DNA insertion was analysed by Southern blotting (Table 2)

TABLE 2
Transgene expression in selected AFQ70.2 lines of tomato

| | RNA | | | | |
|---|---|---|---|---|---|
| Line | | Mature | | | |
| n° | | green | Turning | Red ripe | T-DNA |
| #27 | gsh1: + | gsh1: + | gsh1: − | gsh1: − | 2 copies |
| | gsh2: + | gsh2: + | gsh2: − | gsh2: − | (?) |
| | gst/gpx: + | gst/gpx: + | gst/gpx: − | gst/gpx: − | |
| | gor2: + | gor2: + | gor2: − | gor2: − | |
| #29 | gsh1: + | Gsh1: + | gsh1: + | gsh1: + | Single copy |
| | gsh2: + | gsh2: ? | gsh2: + | gsh2: + | |
| | gst/gpx: + | gst/gpx: + | gst/gpx: + | gst/gpx: + | |
| | gor2: + | gor2: + | gor2: + | gor2: + | |

6. Results—Lettuce

6.1 AFQ70.1 Lines

6.1.1 Expression of Transgenes

Transgene expression was analysed by 3′RACE PCR. Two lines were identified which exhibited expression of the four transgenes, and which appeared to have simple T-DNA integration patterns. These were lines 33 and 39 (Table 3)

TABLE 3
Transgene expression in selected AFQ70.1 lines of lettuce

| | | | | | Estimated copy | |
|---|---|---|---|---|---|---|
| AFQ70.1 | mRNA (3′RACE PCR) | | | | number | |
| line | GOR1 | GSHI | GSHII | GPX | LUC | NPTII |
| WT | − | − | − | − | 0 | 0 |
| 5 | + | + | − | + | 4 | 2 |
| 15 | − | + | − | + | 2 | 2 |
| 16 | − | + | − | + | 0 | 5 |
| 23 | − | + | + | + | 2 | 1 |
| 29 | + | + | − | + | 1 | 1 |
| 32 | + | + | − | + | 3 | 2 |
| 33 | + | + | + | + | 1 | 1 |
| 38 | − | + | + | + | 1 | 1 |
| 39 | + | + | + | + | 2 | 1 |
| 41 | − | + | − | + | 1 | 1 |
| 46 | − | + | + | − | 1 | 1 |
| 51 | − | + | + | + | 1 | 1 |

6.1.2 Glutathione Content

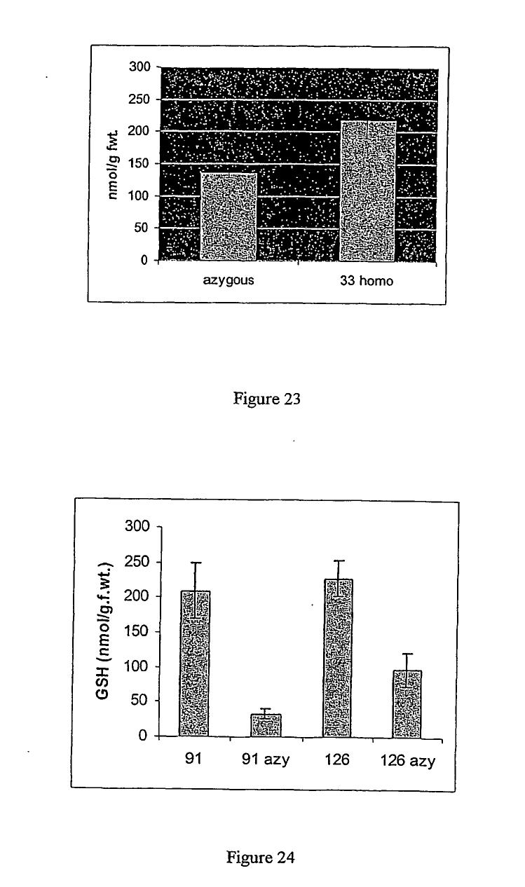

Glutathione was determined in the leaves of AFQ70.1.33, comparing homozygous material with azygous controls. The transgenic material exhibited an approximately 60% elevation in GSH content compared with the azygous control material (FIG. 23)

6.2 AFQ70.2 Lines

6.2.1 Expression of Transgenes

Transformants which had been identified as kanamycin resistant and luciferase positive were allowed to self pollinate. Subsequently, progeny from the T2 generation were screened for transgene expression by western analysis for GSH1, GSH2 and GOR1. No antibody was available for GST/GPX. Lines which expressed all three of the testable gene products were identified.

Samples have been stored for future expression analysis at the mRNA level.

TABLE 4
Western analysis of transgene expression in selected
AFQ70.2 lines

| Line | GSH1 | GSH2 | GOR | GST/GPX |
|---|---|---|---|---|
| 70.2.30 | + | + | + | n/a |
| 70.2.91 | + | + | + | n/a |
| 70.2.36 | + | + | + | n/a |

6.2.2 Glutathione Content

Glutathione was measured in the leaves of selected lines and compared with the levels in azygous control material (FIG. 24).

7. Oxidative Stress

7.1 Response of the Transgenic Tomato Line AFQ70.1.3 to Paraquat Treatment.

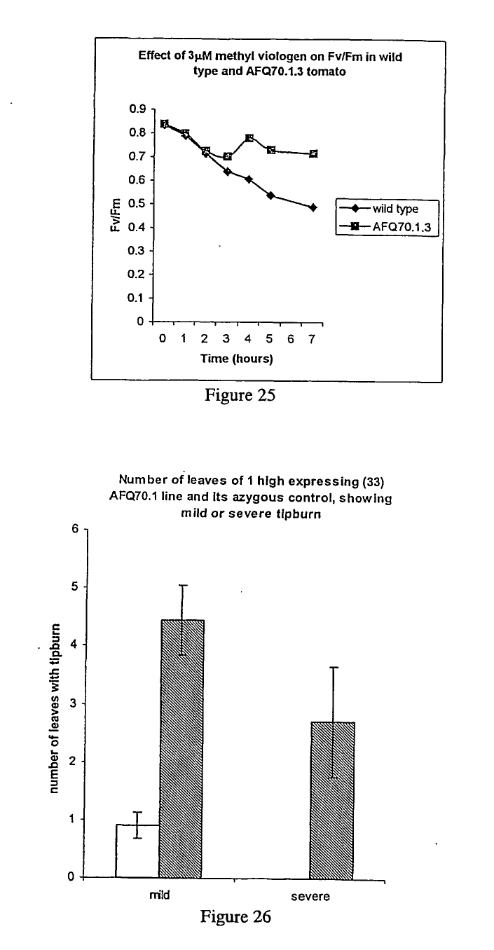

Leaf discs of control (wild-type) tomato and trangenic (AFQ70.1.3) tomato were floated on 3 mM paraquat (methyl viologen) in 1% tween 20 and exposed to light (300 mmol m⁻² s⁻¹). Measurement of the fluorescence parameter Fv/Fm were taken for up to 7 hours. (FIG. 25). The wild-type tomato showed a typical decline in Fv/Fm which was not apparent in the transgenic line. This suggests that the transgenic line shows enhanced tolerance to oxidative stress resulting from the paraquat treatment.

7.2 Tipburn

FIG. 26 illustrate the number of leaves of transgenic lettuce plants expressing AFQ70.1 and their azygous controls showing mild or severe tipburn. Data for mild tipburn for individual plants is shown in Table 5. Both mild and severe tipburn were reduced in the transgenic plants. Number of leaves in each of the high expessing plants showing mild tipburn was 0.9±0.227 (mean±sem) compared to 4.44±0.0.603 (p<0.0001) in the azygous controls. None of the leaves in each of the high expressing plants showed severe tipburn, compared to 2.7±0.957 (p<0.0001) in the azygous controls. In FIG. 26, tipburn open box represents transgenic plants, closed box represents the azygous controls.

TABLE 5
33 HIGH

| plant | 1 | 2 | 3 | 4 | 5 | 6 | 7 | 8 |
|---|---|---|---|---|---|---|---|---|
| Number of leaves with mild | 1 | 1 | 0 | 2 | 0 | 1 | 1 | 1 |
| tipburn | | | | | | | | |
| Number of leaves with | 0 | 0 | 0 | 0 | 0 | 0 | 0 | 0 |
| severe tipburn | | | | | | | | |

33 azy

| plant | 1 | 2 | 3 | 4 | 5 | 6 | 7 | 8 | 9 |
|---|---|---|---|---|---|---|---|---|---|
| Number of leaves with mild | 1 | 5 | 7 | 6 | 3 | 5 | 5 | 5 | 3 |
| tipburn | | | | | | | | | |
| Number of leaves with | 6 | 1 | 3 | 4 | 0 | 8 | 0 | 0 | 2 |
| severe tipburn | | | | | | | | | |

7.3H₂O₂ Levels.

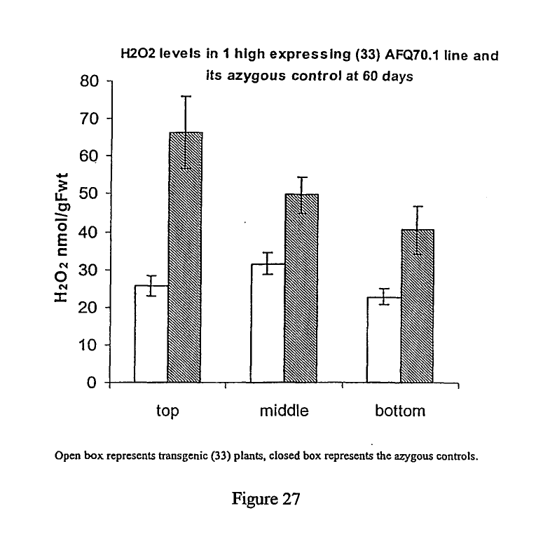

H₂O₂ levels in leaves and leaf disks of transgenic plants was measured using conventional methods (Creissen et al 1999; Plant Cell 11, 1-16) As shown in FIG. 27, H₂O₂ levels in leaves—at top, middle and bottom parts—were significantly reduced in 60 dps (days post sowing) transgenic plants compared to the azygous controls, demonstrating that oxidative stress in these plants will be reduced, enabling the delaying of harvesting of plants. Mean H₂O₂ levels in leaves from the top of the transgenic plants was 25.56±2.72 (mean±sem) compared to 66.22±9.68 (p<0.001) for the azygous controls. Mean H₂O₂ levels in leaves from the middle of the transgenic plants was 31.57±2.86 compared to 49.59±4.91 (p<0.005) for the azygous controls. Mean H₂O₂ levels in leaves from the bottom of the transgenic plants was 22.61±2.16 compared to 40.42±6.19 (p<0.02) for the azygous controls.

7.4 Lipid Peroxidation

Lipid peroxidation of the bottom leaves of lettuces of a high expressing AFQ70.1 line and their azygous controls was measured using conventional methods with a lipid peroxidase assay kit (Bioxytech SA, France)

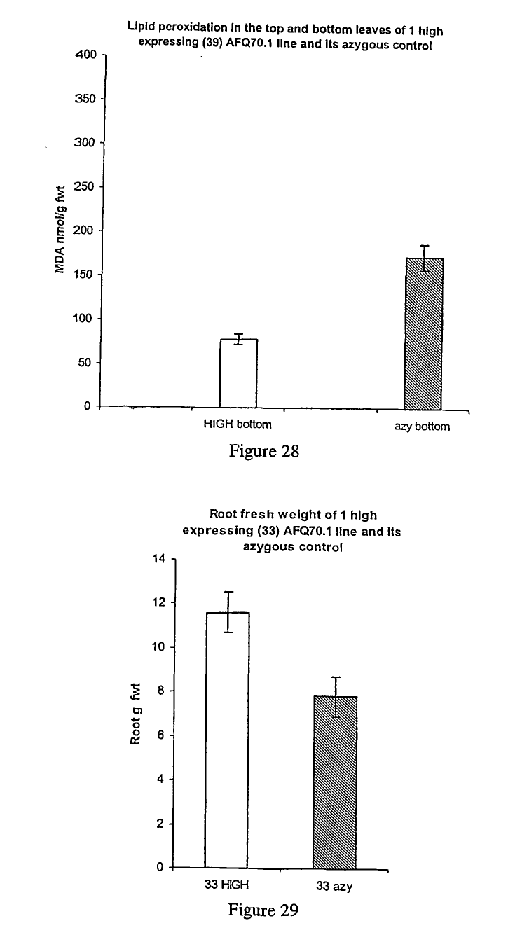

As shown in FIG. 28, lipid peroxidation of the leaves from the transgenic plants was significantly reduced compared to that of the azygous control leaves (p<0.001). This provides further evidence that oxidative stress is reduced in plants of the invention, enabling improvements in the shelf life of the plants. Malondialdehyde (MDA) in the bottom leaves of the transgenic plants was 78.24±5.99 compared to 172.37±14.67 (p<0.005).

7.5 Root Weight

Root mass and head mass was measured and compared between lettuces expressing the AFQ70.1 line and azygous controls. As shown in FIG. 29, lettuces expressing the AFQ70.1 line had significantly greater root weight (11.59±0.90 (mean±sem)) than their azygous controls (7.8±0.92 p<0.02), enabling consequent improvements in water use and nutrient uptake.

There was no significant difference between the head mass of the transgenic and azygous plants. Transgenic lettuces do not bolt as early as their transgenic controls. By delaying bolting of the plants, harvesting can be delayed and the shelf-life of the harvested plant may be prolonged.

1 A stable recombinant multi-gene nucleic acid construct, which comprises:
(i) a gene encoding γ-glutamylcysteine synthetase (EC 6.3.2.2)
(ii) a gene encoding glutathione synthetase (EC 6.3.2.3)

2 A construct as claimed in claim 1 wherein the gene encoding γ-glutamylcysteine synthetase is the gsh1 gene and\or the gene encoding glutathione synthetase is the gsh2 gene.

3 A construct as claimed in claim 1 or claim 2 wherein each of said genes is operably linked to a different promoter such as to enable differential expression of γ-glutamylcysteine synthetase and glutathione synthetase.

4 A construct as claimed in any one of the preceding claims which comprises at least one gene operably linked to a promoter, which gene encodes an enzyme involved in the redox cycling of glutathione between its reduced and its oxidised forms.

5 A construct as claimed in claim 4 which comprises two different genes each operably linked to a promoter, which genes each encode a different enzyme involved in the redox cycling of glutathione between its reduced and its oxidised forms.

6 A construct as claimed in claim 4 or claim 5 wherein the or one enzyme involved in the redox cycling is encoding glutathione reductase (GOR).

7 A construct as claimed in claim 6 wherein the glutathione reductase is plastidial glutathione reductase (GOR1).

8 A construct as claimed in claim 6 wherein the glutathione reductase is cytosolic glutathione reductase (GOR2)

9 A construct as claimed in claim in any one of claims 4 to 8 the or one enzyme involved in the redox cycling is glutathione peroxidase.

10 A construct as claimed in claim 9 wherein the glutathione peroxidase is phospolipid hydroperoxide glutathione peroxidase (phGPX).

11 A construct as claimed in claim 9 wherein the glutathione peroxidase is cytosolic glutathione peroxidase/glutathione-S-transferase (GST/GPX).

12 A construct as claimed in any one of claims 3 to 11 wherein the gene encoding γ-glutamylcysteine synthetase is operably linked to a weaker promoter than the gene encoding glutathione synthetase.

13 A construct as claimed in any one of the preceding claims wherein at least one of the promoters is an inducible promoter.

14 A construct as claimed in any one of the preceding claims wherein each of the promoters is present in the construct as no more than one copy.

15 A construct as claimed in any one of the preceding claims wherein each of the promoters is heterologous to the gene with which it is operably linked.

16 A construct as claimed in claim 15 wherein the:
(i) the gene encoding γ-glutamylcysteine synthetase is operably linked to a Ef1a promoter;
(ii) the gene encoding glutathione synthetase is operably linked to a cauliflower mosaic virus (CaMV) 35S promoter; and optionally
(iii) the GOR gene if present is operably linked to a AtrpL1 promoter; and optionally
(iv) the GPX gene if present is operably linked to a UBQ1 promoter.

17 A construct as claimed in any one of the preceding claims which is a plant binary vector.

18 A vector as claimed in claim 17 comprising selectable genetic markers.

19 A vector as claimed in claim 18 wherein the markers are a firefly luciferase (luc) reporter gene and kanamycin resistance (kan; NPTII).

20 A vector as claimed in any one of claims 17 to 19 which is the pAFQ70-1 plasmid as illustrated in FIG. 13 or the pAFQ70-2 plasmid as illustrated in FIG. 20.

21 A method which comprises the step of introducing the vector of any one of claims 17 to 20 into a plant host cell, and optionally causing or allowing recombination between the vector and the host cell genome such as to transform the host cell.

22 A host cell containing or transformed with a heterologous vector of any one of claims 17 to 20.

23 A method for producing a transgenic plant, which method comprises the steps of:
(a) performing a method as claimed in claim 21
(b) regenerating a plant from the transformed plant cell.

24 A transgenic plant which is obtainable by the method of claim 17, or which is a clone, or selfed or hybrid progeny or other descendant of said transgenic plant,
which in each case includes the plant cell of claim 22 and which express heterologous genes encoding γ-glutamylcysteine synthetase and glutathione synthetase plus optionally one or more heterologous genes encoding enzymes involved in the redox cycling of glutathione between its reduced and its oxidised forms.

25 A transgenic plant as claimed in claim 24 wherein the heterologous genes are expressed in at least two subcellular compartments

26 A transgenic plant as claimed in claim 24 or claim 25 which is selected from the list consisting of: tomato, pepper, aubergine, courgette, lettuce, cabbage, broccoli, ornamentals, potato and yam.

27 A part of propagule from a plant as claimed in any one of claims 24 to 26, which in each case includes the plant cell of claim 22 and which express heterologous genes encoding γ-glutamylcysteine synthetase and encoding glutathione synthetase plus optionally one or more heterologous genes encoding enzymes involved in the redox cycling of glutathione between its reduced and its oxidised forms.

28 A method for providing a plant having enhanced levels of reduced glutathione, which method comprises the steps of performing a method of claim 23 and optionally replicating the transgenic plant and, wherein one or more of the promoters of the vector is an inducible promoter, applying an exogenous inducer of said inducible promoter.

29 A method for providing fruit having enhanced levels of reduced glutathione, which method comprises the steps of performing a method of claim 28 and harvesting fruit from the plant.

30 A method for improving oxidative stress tolerance of a plant; enhancing root development of plant; increasing the post-harvest shelf life of a plant or fruit; delaying the bolting of a plant, which method comprising performing the method of claim 28 or claim 29.

31 A process for producing vector as claimed in claim 20 substantially as described in the Examples 1-3 herein with reference to FIGS. 1 to 20.
